(19) United States
(12) Patent Application Publication
(10) Pub. No.: US 2026/0243317 A1
(43) Pub. Date: Aug. 20, 2026

(54) SWITCHABLE CLUTCH BASED ON SELF-AMPLIFIED FRICTION OF INTERLEAVED LAYERS

(71) Applicant: MASSACHUSETTS INSTITUTE OF TECHNOLOGY, Cambridge, MA (US)

(72) Inventors: Anastasios John HART, Waban, MA (US); Aoyi LUO, Syracuse, NY (US)

(21) Appl. No.: 19/472,445

(22) PCT Filed: Aug. 5, 2024
(86) PCT No.: PCT/US2024/041002

§ 371 Date: Oct. 3, 2025

Related U.S. Application Data
(60) Provisional application No. 63/517,895, filed on Aug. 5, 2023.

(51) Int. Cl. F16D 63/00; A61F 5/02; B25J 9/00; B25J 9/10

(52) U.S. Cl. CPC F16D 63/008; A61F 5/026; B25J 9/0006; B25J 9/1075; F16D 2250/0007

(57) ABSTRACT

A clutch device includes two pluralities of flexible members interleaved with each other. An overlapping region is defined where the flexible members overlap each other. Non-overlapping regions are on opposing sides of the overlapping region. The non-overlapping regions are regions in which the flexible members do not overlap each other. In a locked arrangement, an outer portion of each non-overlapping region extends at a first angle relative to the overlapping region. The first angle causes the flexible members to engage and interlock with each other. The outer portions of can be moved to a second angle different than the first angle to unlock the clutch device and allow the flexible members to disengage with and be separated from each other. The clutch device thus does not require continuous energy input while also having a high locking force that can be rapidly applied or released.

### CROSS REFERENCE TO RELATED APPLICATION

The present disclosure claims priority to and the benefit of U.S. Provisional Patent Application No. 63/517,895, entitled “Switchable clutch based on self-amplified friction of interleaved layers,” filed Aug. 5, 2023, the disclosure of which is incorporated by reference herein in its entirety.

### FIELD

The present disclosure relates to locking mechanisms, and in particular, clutches configured to be selectively engaged and disengaged.

### BACKGROUND

A clutch is a mechanical device that enables switchable engagement. Specifically, a clutch is a locking device that switches between allowing and preventing relative motion between two bodies. This means that when the clutch is engaged, the relative velocity between the two bodies is zero. As one non-limiting example, clutches can enable advanced functions in robotic mechanisms, including variable stiffness and hybrid actuation schemes, where springs or actuators can engage individually, in parallel, or in series. In other non-limiting examples, clutches have found applications in exoskeletons, prosthetics, orthoses, haptics, legged robots, and soft robots. These applications highlight the versatility and potential impact of clutches in advancing the capabilities of robotic systems.

Conventional clutches, such as latches and ratchets, rely on mechanical locking mechanisms. While such devices offer fast actuation, high locking force, and low energy consumption only during switching, these clutches have limitations. For example, these devices can only lock in specific positions and cannot be unlocked while under load. Electromagnetic clutches provide fast actuation and high locking force but are heavy, and often have high power consumption.

Recently, alternative clutch designs, including electroadhesive clutches and layer jamming-based clutches, have been explored. Electroadhesive clutches typically have two conductive electrode layers separated by a dielectric layer. When a voltage is applied between the electrodes, electrostatic attraction generates normal force and, in turn, friction that resists sliding. While electroadhesives are lightweight and power efficient, they require high voltage and are susceptible to electric breakdown of the dielectric layer. In contrast, layer jamming-based clutches involve the use of interleaved layers within a sealed sheath. Clutching is achieved by applying a vacuum within the sheath to compress the layers together, generating a normal force and resultant friction. While effective, vacuum-based interleaved clutches have a lengthy switching time of at least a few seconds due to fluid flow.

An optimized clutch should possess several key characteristics, including having a high locking force, low energy consumption, compactness, cost-effectiveness, robustness, rapid switching time, and being lightweight. Such a clutch would have important implications, such as, for example, improving compact and lightweight exoskeletons and rehabilitation devices, as well as legged robots with reduced energy consumption and motor requirements. To date, such optimized clutches are either non-existent or not able to achieve enough success in these identified key characteristics, nor have such clutches been used in applications such as exoskeletons and robots, or other applications provided for later in the present disclosure for that matter.

Accordingly, there is a need for optimized clutches that possess many or all of the key characteristics while minimizing the drawbacks of previously contemplated clutches.

### SUMMARY

This Summary introduces a selection of concepts in simplified form that are described further below in the Detailed Description. This Summary does not necessarily identifies key or essential features, nor does it limit the scope of the claimed subject matter.

Switchable clutches based on interleaved flexible members (e.g., sheets) are a potential solution to the above-described shortcomings of clutch technology that provide many or all of the key characteristics such as having a high locking force, low energy consumption, compactness, cost-effectiveness, robustness, rapid switching time, and being lightweight. A common demonstration in the physics space involves the concept of “inseparable books,” where the pages of two books are tightly interleaved, requiring a tremendous amount of force to separate them. The high tensile strength of this assembly arises from the amplification of a small normal force exerted by the outermost layers. Such inseparable books do not include convenient means of being separated.

Leveraging aspects of a self-amplified friction mechanism, the clutches disclosed herein include two stacks of layers interleaved together, naturally maintaining a locked state having substantial frictional strength and in which a small normal force is exerted on the stacks via the outermost layers of the stacks. Unlocking the clutch is achieved by eliminating the small normal force exerted by the outermost layers. This can be accomplished, for example, by applying rotation to the distal end of the layers, effectively reversing the direction of the bending force. The present disclosure includes experimental characterizations and analytical modeling of the interleaved clutches to understand the mechanisms of its performance and to demonstrate its integration in a variety of applicable use scenarios. A person skilled in the art, in view of the present disclosures, is able to implement designs involving the clutches described herein across a variety of applications, including but not limited to those described below.

One embodiment of a clutch device includes a first plurality of flexible members, a second plurality of flexible members interleaved with the first plurality of flexible members, at least one overlapping region, and a plurality of non-overlapping regions. The at least one overlapping region is defined in an area in which flexible members of each of the first and second pluralities of flexible members overlap each other, and the plurality of non-overlapping regions are defined in areas on opposing sides of the overlapping region, with the plurality of non-overlapping regions being regions in which the flexible members of each of the first and second pluralities of flexible members do not overlap each other. In a locked arrangement of the clutch device, an outer portion of each of the plurality of non-overlapping regions extends at a first angle relative to a plane defined by a corresponding overlapping region of the at least one overlapping region such that the first and second pluralities of flexible members engage and interlock with each other and prevent the first and second pluralities of flexible members from sliding with respect to each other. The outer portions of each of the plurality of non-overlapping regions are configured to be moved to a second angle relative to the plane defined by the at least one overlapping region to bend the flexible member outwardly, with the second angle being different than the first angle, and such movement causing the clutch device to move into an unlocked arrangement. In the unlocked arrangement, the first and second pluralities of flexible members are disengaged from each other so as to allow the first and second pluralities of flexible members to slide with respect to each other.

The second angle can be greater than the first angle. In at least some embodiments, each flexible member of the first and second pluralities of flexible members can include a first terminal end that overlaps an opposing flexible member of the other of the first and second pluralities of flexible members. Each flexible member of the first and second pluralities of flexible members can further include a second terminal end opposite the first terminal end. Further, a pivot axis can be defined on each flexible member of the first and second pluralities of flexible members about which the respective plurality of flexible members is configured to be rotated. In at least some such embodiments, each flexible member of the first and second pluralities of flexible members can include a distal end portion that can be defined between the pivot axis and the second terminal end. The distal end portion can be bent outwardly relative to a remainder of the flexible member and relative to the outer portion of the flexible member.

The clutch device can further include a third plurality of flexible members, a fourth plurality of flexible members, at least one overlapping region, and a plurality of non-overlapping regions. The first and third pluralities of flexible members can define a first stack of flexible members and the second and fourth pluralities of flexible members can define a second stack of flexible members. The third plurality of flexible members can be interleaved with the fourth plurality of flexible members. The at least one overlapping region can be defined in an area in which flexible members of each of the third and fourth pluralities of flexible members overlap each other, and the plurality of non-overlapping regions can be defined in areas on opposing sides of the overlapping region. The plurality of non-overlapping regions can be regions in which the flexible members of each of the third and fourth pluralities of flexible members do not overlap each other. In the locked arrangement of the clutch device, an outer portion of each of the plurality of non-overlapping regions can extend at a third angle relative to a plane defined by a corresponding overlapping region of the at least one overlapping region such that the third and fourth pluralities of flexible members engage and interlock with each other and prevent the third and fourth pluralities of flexible members from sliding with respect to each other. The outer portions of each of the plurality of non-overlapping regions can be configured to be moved to a fourth angle relative to the plane defined by at least one overlapping region to bend the flexible member outwardly. The fourth angle can be different than the third angle, and such movement can cause the clutch device to move into the unlocked arrangement in which the third and fourth pluralities of flexible members can be disengaged from each other so as to allow the third and fourth pluralities of flexible members to slide with respect to each other.

The clutch device can further include at least one first cord extending through the first and third pluralities of flexible members and at least one second cord extending through the second and fourth pluralities of flexible members. The first and third pluralities of flexible members can be configured to be pulled toward each other and subsequently held in tension via the at least one first cord so as to form the first stack of flexible members. Further, the second and fourth pluralities of flexible members can be configured to be pulled toward each other and subsequently held in tension via at least one second cord so as to form the second stack of flexible members. In at least some such embodiments, the at least one first and second cords can extend through the first, second, third, and fourth pluralities of flexible members adjacent to the pivot axis.

The clutch device can further include a first clamp and a second clamp. The first clamp can extend around the distal end portions of the flexible members of the first and third pluralities of flexible members, and the second clamp can extend around the distal end portions of the flexible members of the second and fourth pluralities of flexible members. The first and second clamps can be configured to clamp the second terminal ends of the flexible members together so as to cause the respective pluralities of flexible members to rotate and move the clutch device into the unlocked arrangement.

In at least some embodiments, the clutch device can further include a first bendable actuation member and a second bendable actuation member. Each flexible member of the first and third pluralities of flexible members can be coupled to the first bendable actuation member, and each flexible member of the second and fourth pluralities of flexible members can be coupled to the second bendable actuation member. In at least some such embodiments, the first and second bendable actuation members can be configured to be bent so as to move opposing outer ends of the first and second bendable actuation members in a direction away from the overlapping region. This can cause the respective pluralities of flexible members to rotate and move the clutch device into the unlocked arrangement.

Another embodiment of a clutch device includes a first plurality of flexible members and a second plurality of flexible members interleaved with the first plurality of flexible members so as to define an overlapping region in an area in which flexible members of the first and second pluralities of flexible members overlap each other. In a locked arrangement of the clutch device, subject to an external load, the first and second pluralities of flexible members can exert a force on the overlapping region so as to cause the first and second pluralities of flexible members to interlock with each other. The first and second pluralities of flexible members are configured to be moved so as to remove the force exerted on the overlapping region and move the clutch device into an unlocked arrangement in which the first and second pluralities of flexible members are disengaged from each other.

The non-overlapping regions can be defined in areas of the first and second pluralities of flexible members in which the flexible members do not overlap each other, and, in a locked arrangement of the clutch device, the first and second non-overlapping regions can exert a force on the overlapping region so as to cause the first and second pluralities of flexible members to interlock with each other and prevent the first and second pluralities of flexible members from sliding with respect to each other.

In at least some embodiments, in the locked arrangement of the clutch device, an outer portion of each non-overlapping region of each flexible member can extend at a first angle relative to a plane defined by a corresponding overlapping region of the flexible member, and the first and second non-overlapping regions of the first and second pluralities of flexible members can be configured to be rotated outwardly relative to the overlapping region. This can increase the first angle, remove the force exerted on the overlapping region, and move the clutch device into the unlocked arrangement in which the first and second pluralities of flexible members are disengaged from each other to allow the first and second pluralities of flexible members to slide with respect to each other.

Each flexible member of the first and second pluralities of flexible members can include a first terminal end that overlaps an opposing flexible member of the other of the first and second pluralities of flexible members. Each flexible member of the first and second pluralities of flexible members can further include a second terminal end opposite the first terminal end, and a pivot axis can be defined on each flexible member of the first and second pluralities of flexible members about which the respective plurality of flexible members can be configured to be rotated. Each flexible member of the first and second pluralities of flexible members can include a distal end portion defined between the pivot axis and the second terminal end, and the distal end portion can be bent outwardly relative to a remainder of the flexible member and relative the outer portion of the flexible member.

The clutch device can further include at least one first cord and at least one second cord. The first cord can be coupled to the first plurality of flexible members closer to the second terminal end than the first terminal end of each flexible member, and the second cord can be coupled to the second plurality of flexible members closer to the second terminal end than the first terminal end of each flexible member. Further, the at least one first and second cords can be interwoven with each flexible member of the respective first and second pluralities of flexible members and can be configured to be tensioned so as to compress the first and second pluralities of flexible members together.

In at least some embodiments, the clutch device can further include a first bendable actuation member and a second bendable actuation member. The first plurality of flexible members can be coupled to the first bendable actuation member, and the second plurality of flexible members can be coupled to the second bendable actuation member. Further, the first and second bendable actuation members can be configured to be bent so as to move opposing outer ends of the first and second bendable actuation members in a direction away from the overlapping region to cause the first and second pluralities of flexible members to rotate and move the clutch device into the unlocked arrangement. In at least some such embodiments, a number of flexible members of the first and second pluralities of flexible members that can be disengaged when the first bendable actuation member is bent such that the opposing outer ends move in a direction away from the overlapping region can depend on an extent of the bending of the first bendable actuation member. Additionally, the extent of the bending of the first bendable actuation member can be selectable such that the number of flexible members that can be disengaged when the first bendable actuation member is bent can be selectable such that a number of flexible members that remain engaged after bending of the first bendable actuation member can also be selectable

The clutch device can further include a first resetting cord member. The first resetting cord member can have a first end coupled to the first plurality of flexible members and a second end opposite the first end coupled to the second plurality of flexible members. The first resetting cord member can be configured to prevent the first and second pluralities of flexible members from sliding away from each other beyond a predetermined stack separation distance. In at least some such embodiments, the first resetting cord member can be resilient such that the first resetting cord member can move the first and second pluralities of flexible members toward each other after the first and second pluralities of flexible members have been slid away from each other.

An embodiment of a method of selectively locking and unlocking a clutch device includes applying a force to at least one of an outer portion of a first plurality of flexible members of a clutch device or an outer portion of a second plurality of flexible members of the clutch device. The first and second pluralities of flexible members are interleaved with respect to each other to cause the interleaved flexible members of the first and second pluralities of flexible members to move from a locked arrangement to an unlocked arrangement. In the locked arrangement, the interleaved flexible members are substantially locked with respect to each other, and in the unlocked arrangement, the interleaved flexible members of the first and second pluralities of flexible members are disengaged from each other.

In at least some embodiments, the method can further include interleaving the second plurality of flexible members with the first plurality of flexible members so as to define at least one overlapping region in an area in which flexible members of the first and second pluralities of flexible members overlap each other. In a locked arrangement of the clutch device, the first and second pluralities of flexible members can exert a force on the at least one overlapping region so as to cause the first and second pluralities of flexible members to interlock with each other.

A plurality of non-overlapping regions of the first and second pluralities of flexible members can be defined in areas on opposing sides of the overlapping region. Further, the applying of the force to cause the interleaved flexible members of the first and second pluralities of flexible members to move to the unlocked arrangement can include moving outer portions of each of the plurality of non-overlapping regions to a second angle relative to a plane defined by the at least one overlapping region to bend the flexible member outwardly. The second angle can be different than the first angle, and such movement can cause the clutch device to move into the unlocked arrangement. In at least some such embodiments, moving the outer portions of each of the plurality of non-overlapping regions can include rotating the outer portions. Each flexible member of the first and second pluralities of flexible members can include a distal end portion defined between a pivot axis and a terminal end of the flexible member. The distal end portion can be bent outwardly relative to a remainder of the flexible member and relative to the outer portion of the flexible member. Further, the rotating of the outer portions can be effected by movement of the distal end portions, which can cause rotation of the first and second pluralities of flexible members about the respective pivot axis.

The interleaving of the second plurality of flexible members with the first plurality of flexible members can include sequentially and alternatingly stacking respective flexible members of the first and second pluralities of flexible members so as to form an interleaved stack of first and second flexible members. In at least some embodiments, the method can further include forming the first and second pluralities of flexible members via at least one of additive manufacturing or 3D printing.

### BRIEF DESCRIPTION OF THE DRAWINGS

The following Detailed Description references the accompanying drawings which form a part this application, and which show, by way of illustration, specific example implementations, in which:

FIG. 1 is a schematic front view of one embodiment of a clutch device, showing interleaved sheets of opposing stacks of sheets in a locked state;

FIG. 2 is a schematic front view of the clutch device of FIG. 1, showing the interleaved sheets of the opposing stacks of sheets in an unlocked state in which distal end portions of the sheets have been rotated to unlock the clutch device;

FIG. 3A is a front view of the clutch device of FIG. 1, showing the interleaved sheets of the opposing stacks of sheets in a locked state, and showing that opposing upper and lower stacks of sheets are slightly spaced apart;

FIG. 3B is a top view of the clutch device of FIG. 3A;

FIG. 4A is a front view of the clutch device of FIG. 3A, showing the interleaved sheets of the opposing stacks of sheets in a locked state, and showing that the opposing stacks can include clamps arranged near distal end portions of the sheets that bring the distal end portions of upper and lower stacks of sheets together;

FIG. 4B is a front view of the clutch device of FIG. 4A, showing the interleaved sheets of the opposing stacks of sheets in an unlocked state in which distal end portions of the sheets have been rotated to unlock the clutch device by moving the clamps toward the respective terminal ends of the sheets;

FIG. 5A is a top view of the clutch device of FIG. 4A, showing the clutch device in the locked state;

FIG. 5B is a top view of the clutch device of FIG. 4B, showing the clutch device in the unlocked state;

FIG. 5C is a schematic front view of the distal end portions of the upper and lower stacks of the clutch device of FIG. 3A, showing a cord that can be tightened to bring the distal end portions of the upper and lower stacks toward each other;

FIG. 6A is a schematic front view of another embodiment of a clutch device, showing interleaved sheets of opposing stacks of sheets being interleaved with each other in a half-stack arrangement and in a locked state;

FIG. 6B is a schematic front view of the clutch device of FIG. 6A, showing interleaved sheets of the opposing stacks of sheets being moved from the locked state to an unlocked state via rotation of distal end portions of the sheets;

FIG. 7A is a graph showing measured locked state frictional strength (Fₒₙ) versus separation distance (L_{f}) for different numbers of layers of sheets (2M), where the dashed lines represent modeling results corresponding to the experiments disclosed herein;

FIG. 7B is a graph showing measured unlocking state frictional strength (F_{off}) as a function of separation distance (L_{f}) for different numbers of layers (2M);

FIG. 7C is a graph showing measured force (F) versus displacement (δ) of the clutch when switched from locking state under load to unlocking state;

FIG. 7D is a graph showing measured locked state frictional strength (Fₒₙ) versus holding time (t) of the clutch when loaded to 300 N with different loading rate (Δd) and then held the displacement;

FIG. 7E is a graph showing switchability of the clutch (Fₒₙ/F_{off}) versus separation distance (L_{f}) for different numbers of layers (2M);

FIG. 7F is a graph showing measured frictional strength for various initial separation distances, and showing measured locked state frictional strength (Fₒₙ) as a function of separation distance (L_{f}) for different numbers of layers and initial separation distances;

FIG. 7G is a graph showing measured frictional strength for various initial separation distances, and showing measured unlocked state frictional strength (F_{off}) as a function of separation distance (L_{f}) for different numbers of layers and initial separation distances;

FIG. 8A is a schematic front view of the clutch device of FIG. 1 in the locked state and the unlocked state, showing horizontal traction (Tₙ) and normal force (fₙ) applied to each layer;

FIG. 8B is a graph of the clutch device of FIG. 8A, showing horizontal traction (Tₙ) distribution within the stack for different numbers of layers (2M) in the locked state;

FIG. 8C is a graph of the clutch device of FIG. 8A, showing horizontal traction (Tₙ) distribution within the stack for various end rotation angles (θ) when the normal force (Tₙhₙ/L_{f}+fₙ) points to a centermost layer;

FIG. 8D is a graph of the clutch device of FIG. 8A showing unlocked state frictional strength (F_{off}) as a function of end rotation angle (θ) for different numbers of layers (2M), where solid lines represent results for θ≤θ_{c}, and dashed lines represent results for θ>θ_{c} under the condition of the interleaved assembly maintaining a tightly packed configuration and results for θ>θ_{c} under the condition of no effective confinement;

FIG. 8E is a further schematic front view of the clutch of FIG. 8A in the locked state and the unlocked state, showing horizontal traction (Tₙ) and normal force (fₙ) applied to the nth layer;

FIG. 8F is a graph of the clutch device of FIG. 8E showing a critical end rotation angle (θ_{c}) as a function of layer number (M) for different separation distances (L_{f}), and indicating that the horizontal traction at the centermost layer becomes zero;

FIG. 8G is a graph of the clutch device of FIG. 8E showing horizontal traction (Tₙ) distribution within the stack for various end rotation angle (θ) when the normal force (Tₙhₙ/L_{f}+fₙ) points to the outermost layer;

FIG. 8H is a graph showing strain energy difference of the clutch device of FIG. 8E between the unlocked and locked states (ΔU) as a function of end rotation angle (θ) for different layer numbers (2M), where solid lines represent results for θ≤θ_{c}, dashed lines represent results for θ>θ_{c} under the condition of the interleaved assembly maintaining a tightly packed configuration, and a dash-dotted line represents results for θ>θ_{c} under the condition of no effective confinement;

FIG. 8I is a further schematic front view of the clutch of FIG. 8A that includes a cover in the locked state and the unlocked state, showing horizontal traction (Tₙ) and normal force (fₙ) applied to each layer, and showing a gap δ between the assembly and the cover in the locked state and the unlocked state with the assembly contacting the cover;

FIG. 8J is a graph of the clutch of FIG. 8I showing horizontal traction (Tₙ) distribution within the stack for various gap distances (δ) when the assembly contacts the cover in the unlocked state;

FIG. 8K is a graph of the clutch of FIG. 8I showing the critical gap distance (δ_{c}) that the contact force between the outermost layer and the cover becomes zero as a function of layer number (M) for various end rotation angles (θ);

FIG. 8L is a graph of the clutch of FIG. 8I showing horizontal traction (Tₙ) distribution within the stack for various end rotation angles (θ) and gap distances (δ) such that the contact force of the assembly with the cover is exactly zero in the unlocked state;

FIG. 9 is a diagram comparing clutch devices of the present disclosure and prior clutch devices as a function of Force/Power Consumption versus Force/Mass, showing the disclosed clutch devices and a mechanical latch represented as dashed lines because they do not require continuous power consumption, showing an “x” to denote a linear clutch, and an “∘” to denote a rotary clutch, with force calculated by dividing torque by the radius of the clutch plate;

FIG. 10A is a schematic front view of another embodiment of a clutch device, showing interleaved sheets of opposing stacks of sheets in a locked state, and showing that the clutch device can include a bendable actuation member configured to rotate distal end portions of the sheets to unlock the clutch device;

FIG. 10B is a schematic front view of the clutch device of FIG. 10A, showing the interleaved sheets of the opposing stacks of sheets in an unlocked state, and showing that the bendable actuation member has been bent outwardly to rotate the distal end portions of the sheets and unlock the clutch device;

FIG. 11A is a schematic front view of the clutch device of FIG. 10A, showing the clutch device in a fully locked state and how the clutch device provides precise control of unlocking a number of sheets that are engaged or disengaged with each other,

FIG. 11B is a schematic front view of the clutch device of FIG. 11A, showing the clutch device in a partially locked state and how the clutch device provides precise control of unlocking a number of sheets that are engaged or disengaged with each other via the bendable actuation member;

FIG. 11C is a schematic front view of the clutch device of FIG. 11A, showing the clutch device in a fully unlocked state and how the clutch device provides precise control of unlocking a number of sheets that are engaged or disengaged with each other via the bendable actuation member;

FIG. 12A is a graph showing locking strength versus separation distance that can be applied to the clutch device of FIG. 10A;

FIG. 12B is a graph showing the locking strength versus separation distance of a prior clutch device, the clutch device being a five (5) layer clutch based on electroadhesion, and the figure showing the results for the clutch with zero (0) through five (5) layers engaged;

FIG. 12C is a perspective top view of the single layer electroadhesive clutch device represented in the graph of FIG. 12B;

FIG. 13A is a schematic front view of yet another embodiment of a clutch device, showing interleaved sheets of opposing stacks of sheets in a locked state, and showing the interleaved sheets of opposing stacks arranged on a top surface of a platform;

FIG. 13B is a schematic front view of the clutch device of FIG. 13A, showing the interleaved sheets of the opposing stacks of sheets in an unlocked state;

FIG. 13C is a schematic front view of the clutch device of FIG. 13A, showing the interleaved sheets of one of the stacks arranged on a top surface of a first platform and the other of the stacks arranged on a top surface of a second platform, the first and second platforms being movable relative to each other;

FIG. 13D is a schematic front view of the clutch device of FIG. 13C, showing the stacks being moved away from each other via movement of the first and second platforms away from each other;

FIG. 14 is a front view of the clutch device of FIG. 13A, showing the interleaved sheets of the opposing stacks of sheets in a locked state, and showing that the clutch device has remained in the locked state after cords holding the sheets in tension have been pulled toward the top surface of a curved platform;

FIG. 15A is a front view of one embodiment of a back brace posture corrector that includes the clutch device of FIG. 3A, the connector being worn by a human, and the clutch device not being visible because it is disposed within a tubing cover;

FIG. 15B is a front view of the back brace posture corrector of FIG. 15A with the tubing cover, and a sliding resetting cord member, removed such that the clutch device is visible;

FIG. 15C is a perspective view of the clutch device of FIG. 15B actuated by a motor and a claw, the clutch device being in a locked state;

FIG. 15D is a perspective view of the clutch device of FIG. 15B actuated by the motor and the claw, the clutch device being in an unlocked state with the claw closed;

FIG. 15E is a front view of the clutch device of FIG. 15B, the clutch device including the sliding resetting cord member;

FIG. 15F is a schematic front view of another embodiment of a clutch device, this clutch device is completely separated without a resetting cord member;

FIG. 15G is a schematic front view of the clutch device of FIG. 15F, showing how the interleaved sheets can engage each other in a stochastic manner without having a resetting cord member;

FIG. 16 shows perspective views of one embodiment of a plurality of modular robots, the modular robots including an embodiment of a clutch device such as the clutch device of FIG. 15G that does not include a resetting cord member and can be utilized to connect and disconnect the modular robots;

FIG. 17 is a schematic illustration of another embodiment of a plurality of modular robots and the clutch device of FIG. 10A, showing how the clutch device that includes bendable actuation members and does not include a resetting cord member can be utilized to connect and disconnect the modular robots;

FIG. 18A is a schematic perspective view of one embodiment of a haptic glove having the clutch device of FIG. 6A integrated into it, the clutch device being in a half-stack implementation;

FIG. 18B is a schematic top view of another embodiment of a haptic glove having the clutch device of FIG. 3A integrated into it, the clutch device being in a full-stack implementation;

FIG. 19A is a schematic front view of one embodiment of an exoskeleton having the clutch device of FIG. 6A integrated into it, the clutch device being in a half-stack implementation;

FIG. 19C is a schematic front view of another embodiment of an exoskeleton having the clutch device of FIG. 3A integrated into it, the clutch device being in a full-stack implementation;

FIG. 20A is a schematic side view of a human user lifting an object and the forces acting on a body of the user during this motion;

FIG. 20B is a side view of an exoskeleton attached to a human user to aid the human user in lifting an object;

FIG. 20C is a schematic side view of one embodiment of an exoskeleton having the clutch device of FIG. 6A integrated into it, the clutch device being in a half-stack configuration;

FIG. 20D is a schematic side view of another embodiment of an exoskeleton having the clutch device of FIG. 3A integrated into it, the clutch device being in a full-stack configuration;

FIG. 21 is a schematic front view of one embodiment of a “soft finger” having the clutch device of FIG. 3A integrated into it, the clutch device being in a full stack configuration;

FIG. 22A shows a perspective view of one embodiment of a damping seat belt having the clutch device of FIG. 3A integrated into it;

FIG. 22B shows a perspective view of one embodiment of a helmet as a damper having the clutch device of FIG. 3A integrated into it;

FIG. 22C shows a perspective view of one embodiment of an ankle exoskeleton configured as a clutch having the clutch device of FIG. 3A integrated into it; and

FIG. 22D shows a perspective view of one embodiment of a flexion muscle rehabilitation device configured as a brake having the clutch device of FIG. 3A integrated into it.

### DETAILED DESCRIPTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present disclosure is defined solely by the claims. The features illustrated or described in connection with one exemplary embodiment may be combined with the features of other embodiments. Such modifications and variations are intended to be included within the scope of the present disclosure. Further, the present disclosure provides some illustrations and descriptions that include prototypes, bench models, and/or schematic illustrations of various ways by which clutch devices operate and/or can be used in conjunction with other objects and/or applications (e.g., back brace posture correctors, seat belts, neck guards and helmets, muscle rehabilitation devices, haptic gloves, exoskeletons, “soft fingers,” modular robots, and other medical devices and robotic devices, among other uses provided for herein or otherwise derivable from the present disclosures). A person skilled in the art will recognize how to rely upon the present disclosure to integrate and/or implement the clutch devices, and related methods of operating the same, provided for herein into various products, devices, systems, and applications. To the extent features are described as being disposed on top of, below, next to, etc. such descriptions are typically provided for convenience of description, and a person skilled in the art will recognize that, unless stated or understood otherwise, other locations and positions are possible without departing from the spirit of the present disclosure.

Unless otherwise defined, all technical terms used herein have the same meaning as commonly understood by one of ordinary skill in the art to which this disclosure belongs. Additionally, like-numbered components across embodiments generally have similar features unless otherwise stated or a person skilled in the art would appreciate differences based on the present disclosure and his/her knowledge. Accordingly, aspects and features of every embodiment may not be described with respect to each embodiment, but those aspects and features are applicable to the various embodiments unless statements or understandings are to the contrary.

The clutch devices described herein, such as clutch devices, 10′, 10″, 210, 310, can include two stacks of flexible members or layers, such as sheets, interleaved together in an initial locked state, in particular due to a normal force being imparted on an overlapping region of the flexible members by non-overlapping regions of the outer flexible members. Via movement or rotation of the non-overlapping regions of the flexible members, this normal force can be removed and the two stacks of layers can be unlocked. This can be accomplished, for example, by applying rotation to the distal ends of the flexible members.

The clutch devices described herein, such as clutch devices, 10′, 10″, 210, 310, provide many or all of the key characteristics of clutches described above, such as having a high locking force, low energy consumption, compactness, cost-effectiveness, robustness, rapid switching time, and being lightweight.

### Clutch Devices

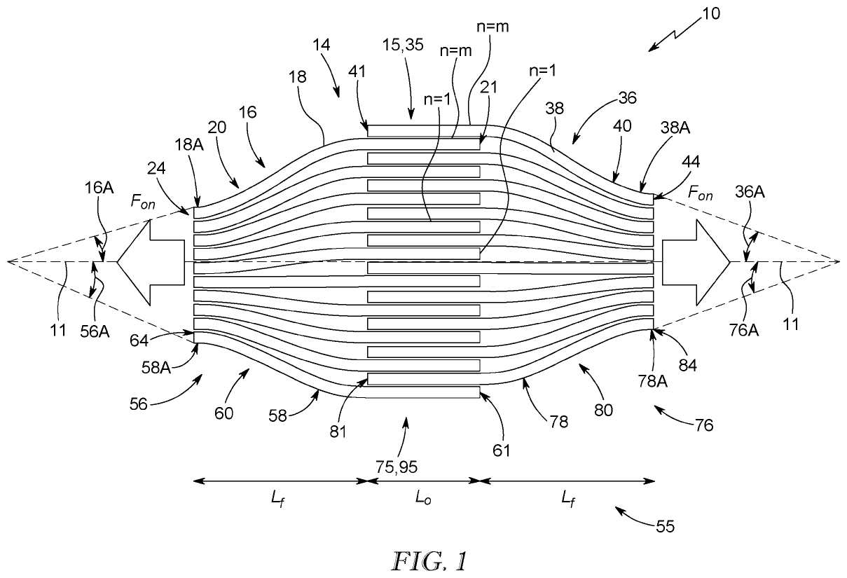

FIG. 1 illustrates one embodiment of a clutch device 10 that includes two stacks 14, 55 of flexible members, as shown sheets 18, 38, 58, 78, interleaved with each other, naturally maintaining a locked state (also referred to as a “locked arrangement”) having substantial frictional strength. The term “flexible member,” as used herein, can refer to any elongated, flexible material or structure that is capable of being interleaved and flexed. While the present disclosure often refers to and illustrates “sheets” in this context, reference to and use of the term “sheets” is in no way limiting. Any reference to a “sheet” can generally be applicable to any “flexible member” provided for herein or otherwise known to those skilled in the art. Non-limiting examples of flexible members that can be used in conjunction with the present disclosures include sheets, fibers, and/or leaves. Such flexible members can typically be disposed in a bundle, thus forming a bundle of interleaved, elongated flexible members.

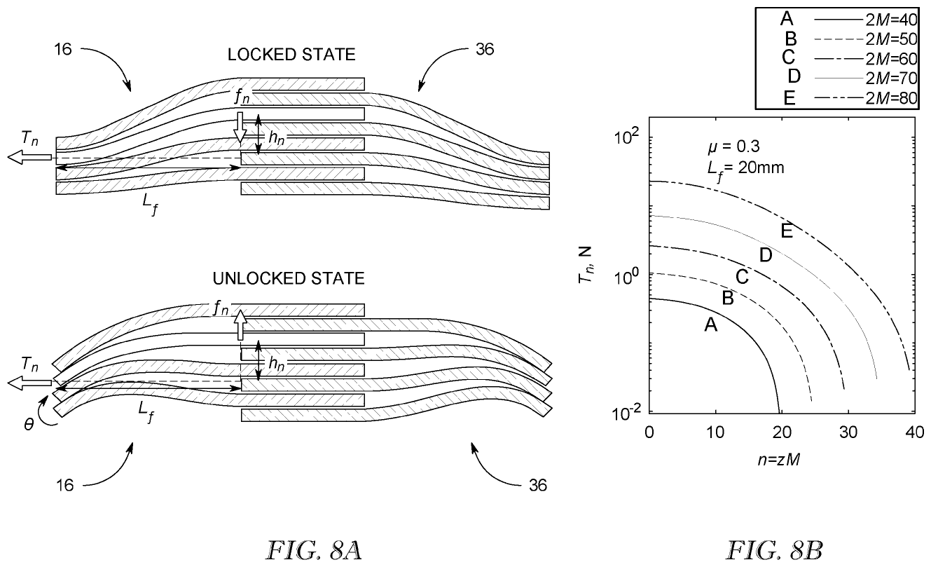

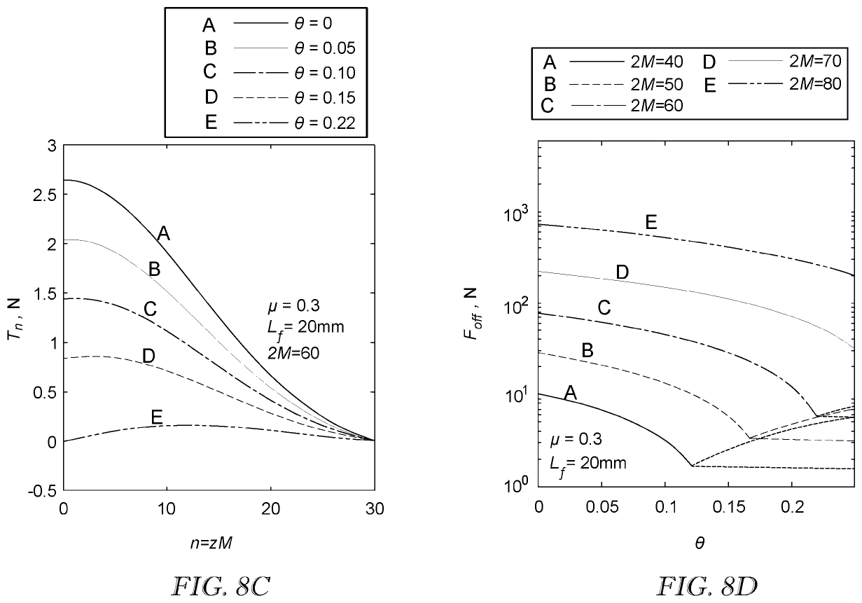

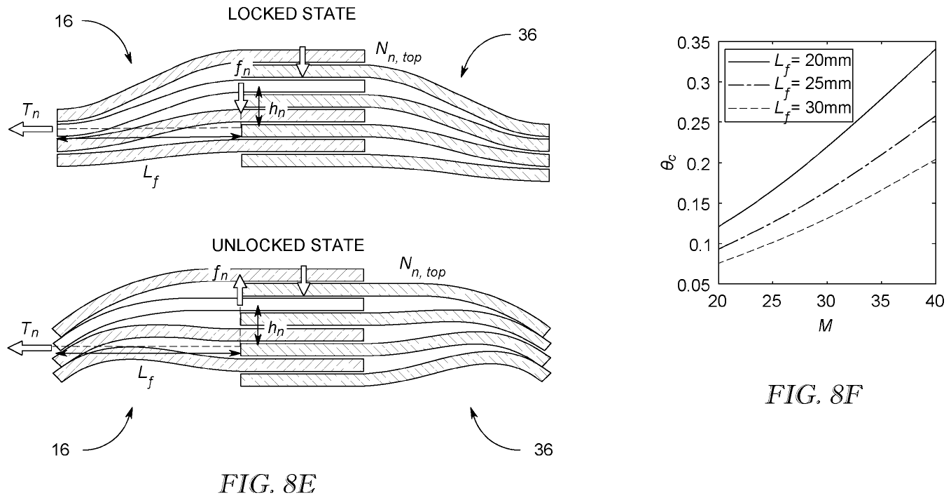

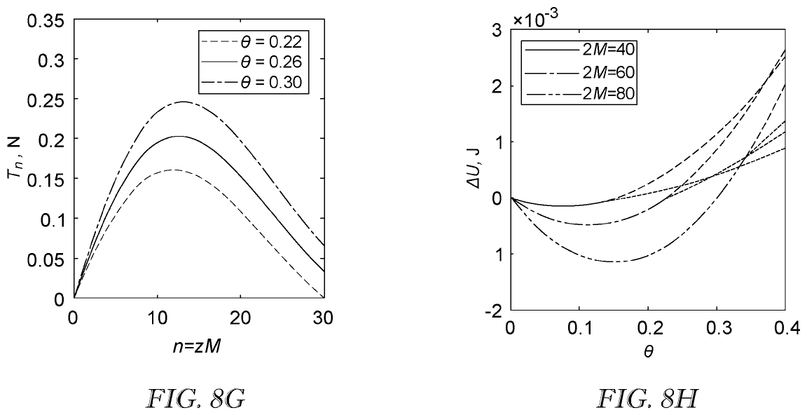

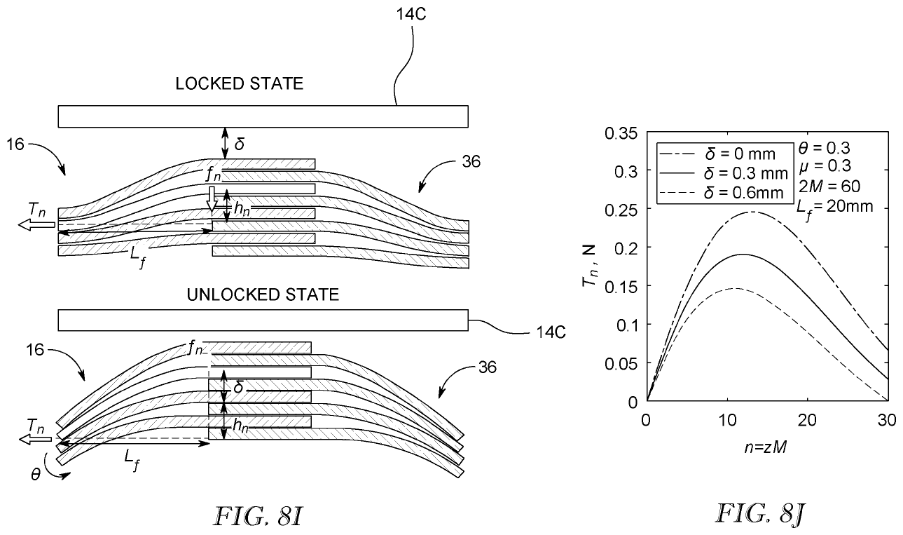

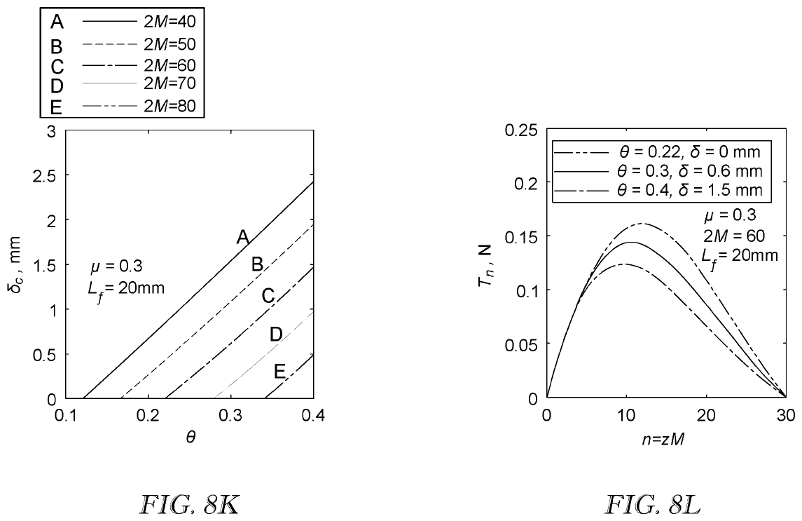

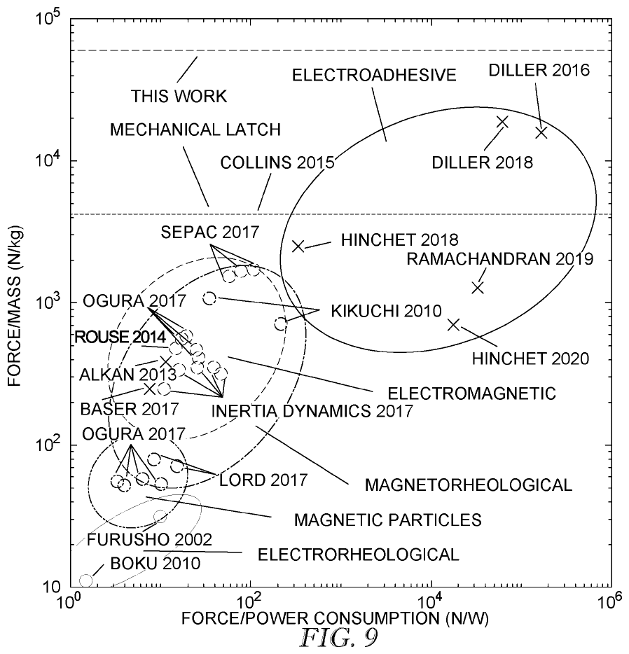

As shown in FIG. 1, the sheets 18, 38, 58, 78 are arranged in a manner such that in the locked state a small normal force (e.g. normal force fₙ in FIGS. 8A, 8E, 8I, which applies to the embodiment shown in FIG. 1) is exerted on the stacks 14, 55 via the outermost sheets 18, 36, 58, 78 of the stacks 14, 55. The clutch device 10 can be moved into an unlocked state (also referred to as an “unlocked arrangement”) by reducing and/or eliminating the small normal force exerted by the outermost sheets 18, 38 58, 78 of the stacks 14, 55. This force can be removed in a variety of ways, for example by applying a rotational force to distal end portions 22, 42, 62, 82 of the sheets 18, 36, 58, 78, effectively reversing the direction of the normal force to move the clutch device 10 into the unlocked state.

The design of the clutch device 10, as well as other clutch devices disclosed herein, including but not limited to clutch devices, 10′, 10″, 210, 310, or otherwise derivable from the present disclosure, are designed and/or can be designed to have a high locking force, low energy consumption, compactness, cost-effectiveness, robustness, rapid switching time, and/or be lightweight.

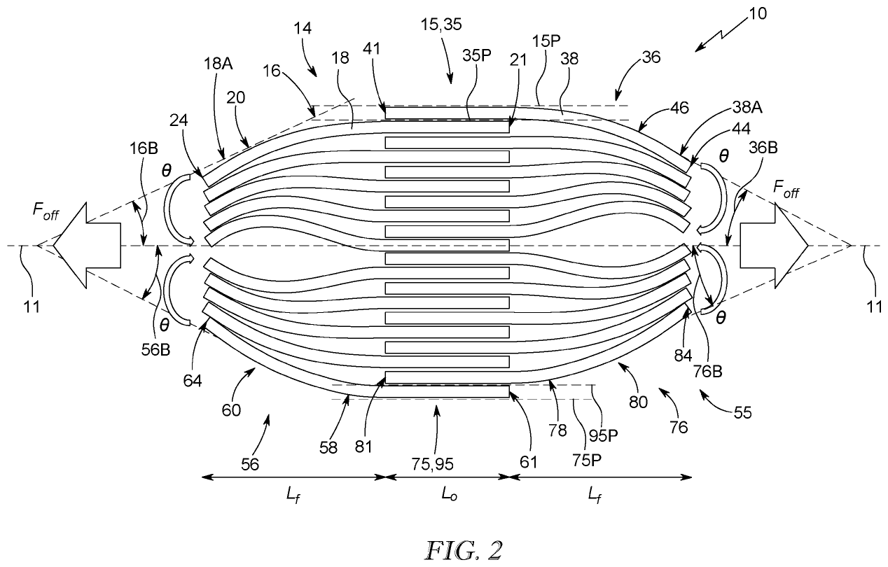

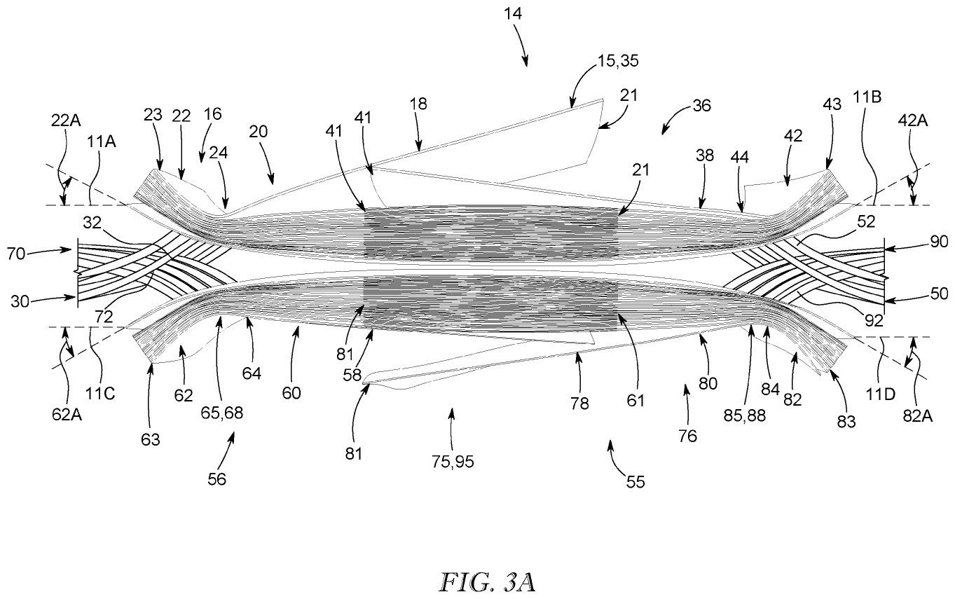

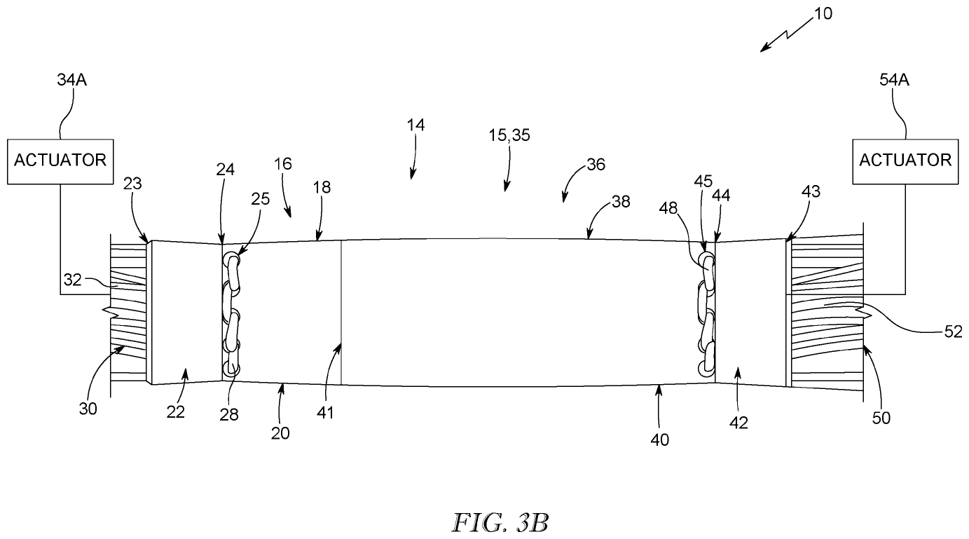

A person skilled in the art will understand that the schematic views and illustrations included in the present disclosure, in particular, for example, FIGS. 1, 2, 5C-6B, 8A, 8E, and 8I, although described herein as applying to specific embodiments of the disclosure, illustrate concepts and working principles that are applicable to all embodiments of clutch devices described herein and any other clutch devices that could be envisioned based on the present disclosure. For example, although the schematic drawings of FIGS. 1 and 2 are shown and described as being part of the embodiment shown FIGS. 3A-5B, the concepts shown in FIGS. 1 and 2 (for example, the concept of the direction of the normal force, the concept of the angle of the non-overlapping regions in locked versus unlocked states, the concept of rotation of the outer portions of the non-overlapping region to cause removal of the normal force, and the like) all apply to the other embodiments described herein.

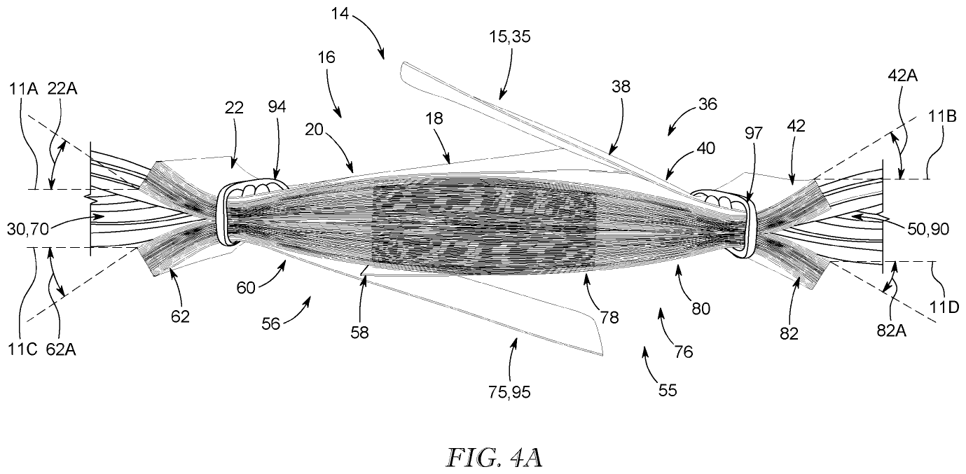

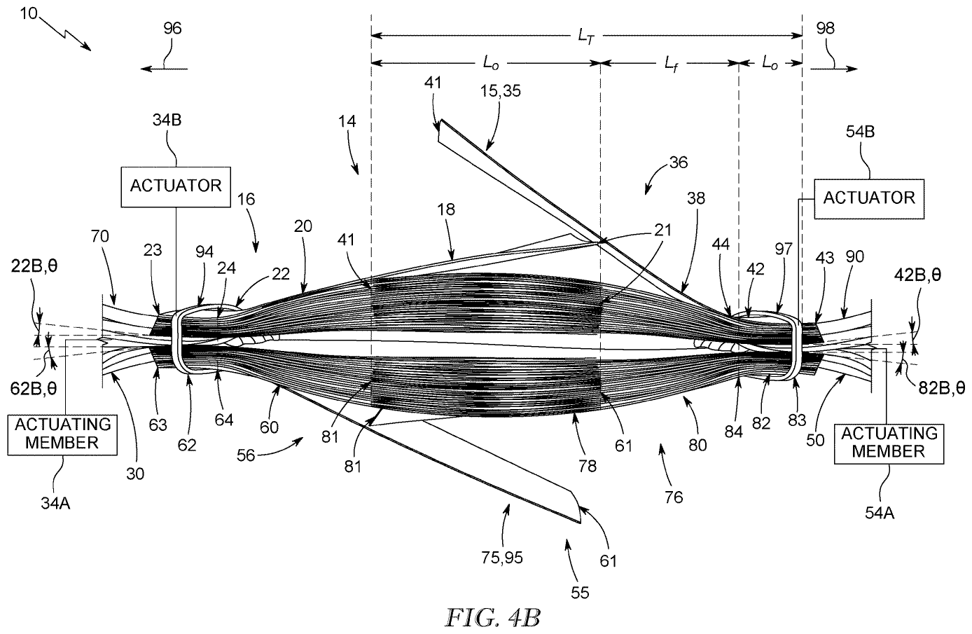

In at least some embodiments of the present disclosure, the clutch device 10 includes a first plurality of sheets 16 and a second plurality of sheets 36 interleaved with the first plurality of sheets 16, as shown schematically in FIGS. 1 and 2, and in greater detail in FIGS. 3A, 4A, and 4B. The clutch device 10 can further include a third plurality of sheets 56 and a fourth plurality of sheets 76 interleaved with the third plurality of sheets 56. It is noted that the term “sheets” is also referred to as “layers” herein, and, as noted above, “sheets” is but one example of flexible members that can be used to form the clutch device 10, and other clutch devices disclosed herein or otherwise derivable from the present disclosures.

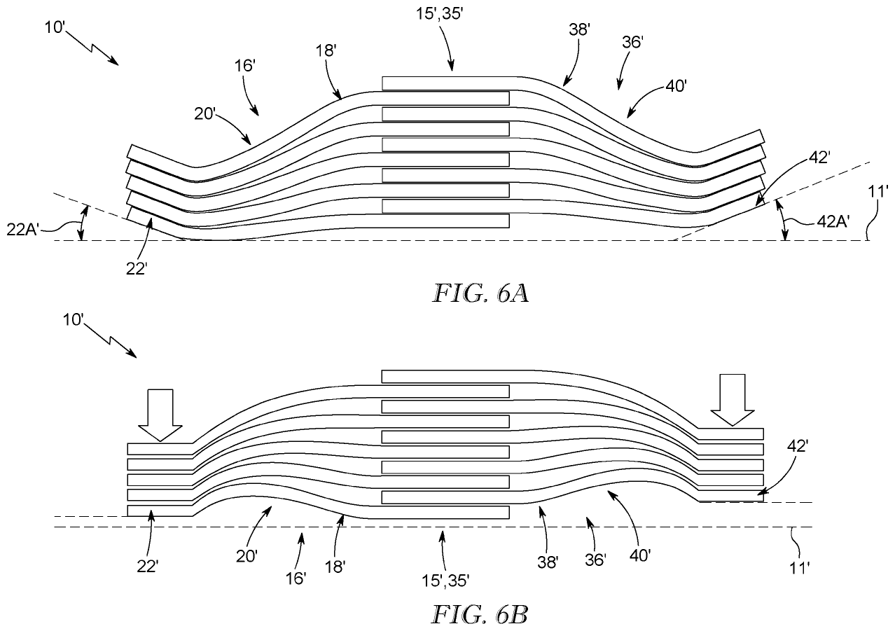

As will be described in greater detail below, the clutch device 10 can be formed as a “full-stack” arrangement (e.g., FIGS. 1-5B) in which the first and third pluralities of sheets 16, 56 form a first stack 14 of sheets and the second and fourth pluralities of sheets 36, 76 form a second stack 55 of sheets. In such embodiments, the clutch device 10 can have a central longitudinal axis 11 extending centrally between the first and second pluralities of sheets 16, 36 and the third and fourth pluralities of sheets 56, 76, as shown in FIGS. 1 and 2. In some other embodiments, the clutch device 10′, 310 can be formed as a “half-stack” arrangement (e.g., FIGS. 6A, 6B, and 13A-14), in which the clutch device 10′, 310 only includes two pluralities of sheets 16′, 36′, 316, 336 that form the “stacks” of interleaved sheets (i.e. stacks 14′, 55′). The “half-stack” arrangement operates to unlock the clutch device 10′ in the same manner as the “full-stack” arrangement, in particular via rotation of the distal end portions 22′, 42′ which in turn rotates the pluralities of sheets 16′, 36′ so as to unlock the clutch device 10′. In such embodiments, the clutch device 10′ can have a similar central longitudinal axis 11′ extending below the first and second pluralities of sheets 16′, 36′.

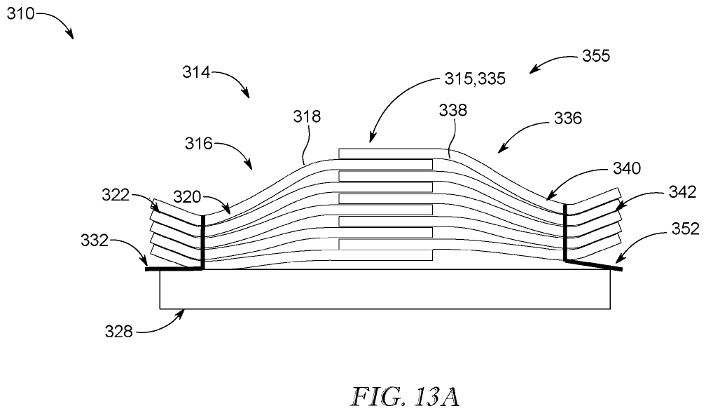

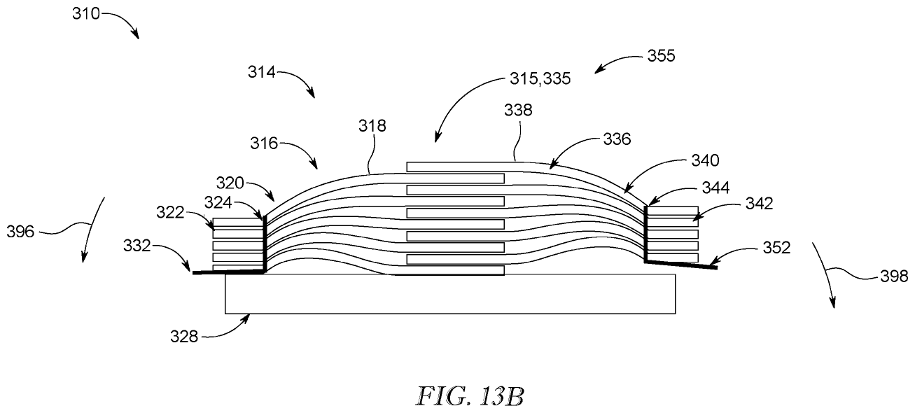

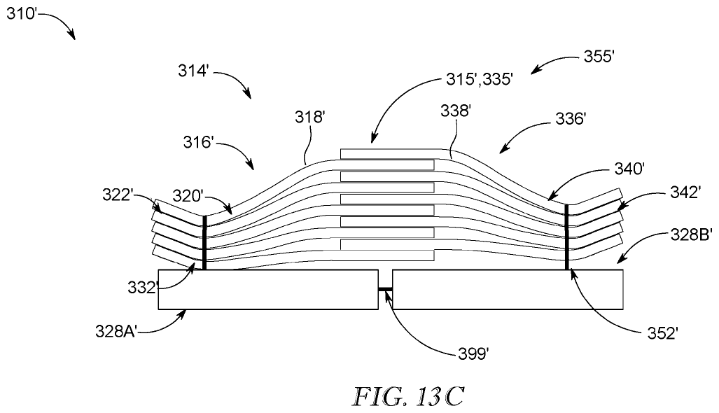

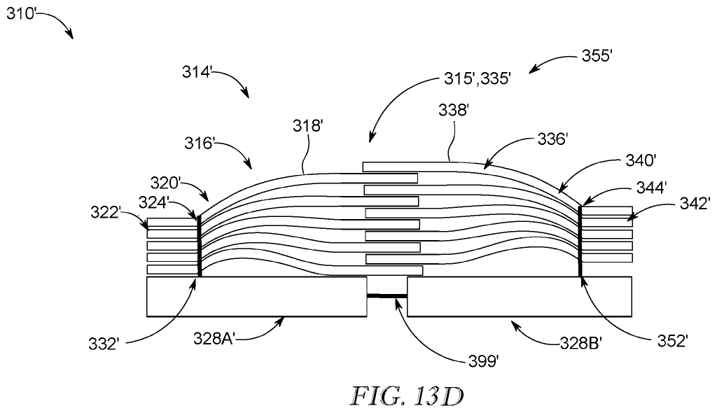

Each of the “half-stack” and “full-stack” arrangements of the clutch device 10, 10′ provide unique advantages. By way of example, an advantage of a “full stack” arrangement is that no platform is required, as opposed to typical “half stack” arrangements, which generally require a platform (see, e.g., a surface or platform 328 in FIG. 13A). More specifically, and with reference to FIG. 13A, for a “half-stack” arrangement, the pluralities of sheets 316, 336 apply the normal force to the platform 328, while the reactionary normal force from the platform 328 is applied to the sheets 316, 336, thus creating the friction force required to keep the stacks 314, 355 of the sheets 316, 336 locked. The platform 328 should be sufficiently rigid so as to be able to apply this reactionary normal force. The “full-stack” arrangement, on the other hand, need not be arranged on a surface. Instead, the normal forces of the upper pluralities of sheets 16, 36 and the lower pluralities of sheets 56, 76 can cancel each other out.

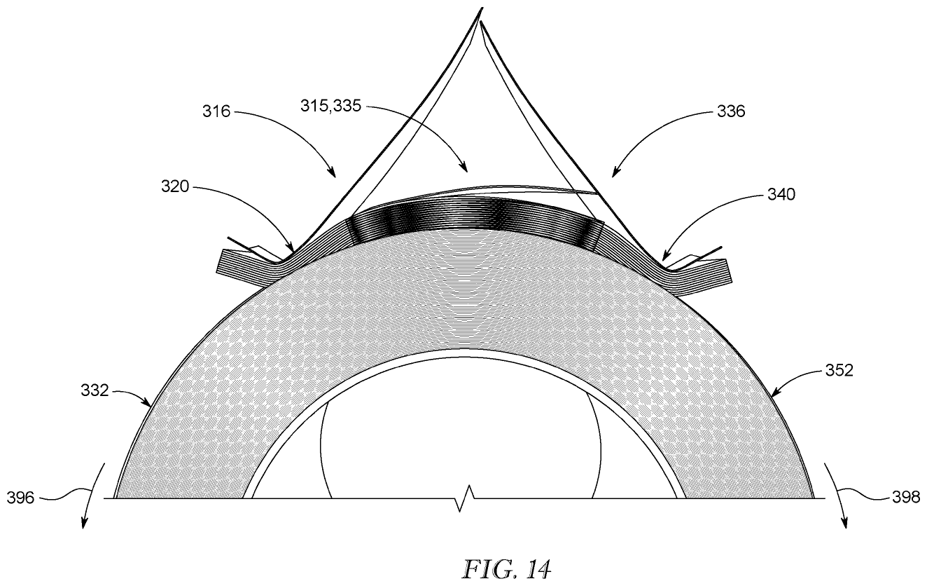

By way of further example, an advantage of a “half-stack” arrangement is that space is only required on one side for stacks to open and unlock. As shown in FIGS. 3A-5B, a “full stack” arrangement typically requires space on both sides for the stacks 14, 55 to open and achieve optimal performance. The “half-stack” arrangement, on the other hand, only requires space on one side for the stacks 16, 36 to open and unlock. Additionally, the “half-stack” can bend to adapt to a curved surface and still function effectively, as shown in FIG. 14. This makes the “half-stack” arrangement ideal for situations where it needs to be placed on a surface that might bend significantly on one side while having an open space on the other side for unlocking.

As shown schematically in FIGS. 1 and 2, and in greater detail in FIGS. 3A-5B, a “full-stack” arrangement of the clutch device 10 includes the first and third pluralities of sheets 16, 56 of the first stack 14 interleaved with the second and fourth pluralities of sheets 36, 76 of the second stack 55. In other words, the sheets 18, 58 of the first stack 14 and the sheets 38, 78 of the second stack 55 are layered on top of another in alternating fashion, as more clearly shown in FIGS. 1 and 2. The sheets 18, 38, 58, 78 of each stack 14, 55 may also be considered to be interdigitated with each other. A person skilled in the art will understand that the term “interleaved” need not always refer to one-to-one alternation, but may also refer to having two sheets between adjacent sheets, three sheets between adjacent sheets, and similar configurations. Moreover, a person skilled in the art will understand that not every single sheet in the stacks 14, 55 must be interleaved, so long as enough sheets are interleaved to lock the stacks 14, 55. For completely non-interleaved sheets, it is expected that the locking force will be negligible. In some embodiments, stacks 14, 55 including, for example, two sheets between adjacent sheets, three sheets between adjacent sheets, and similar configurations, the sheets between adjacent sheets can be regarded as an equivalent thick sheet. Using two sheets between adjacent sheets as an example, if there are 60 sheets, each 0.1 mm thick, this configuration can be considered equivalent to 30 sheets each 0.2 mm thick. The performance can then be calculated by applying these parameters to the model equation.

Illustratively, each plurality of sheets 16, 36, 56, 76 can include a stack of identically formed sheets 18, 38, 58, 78, as can be seen in FIGS. 3A-5B. The sheets 18, 38, 58, 78 being formed to be identical or substantially identical to each, can aid in ensuring that the various forces acting on the stacks 14, 55 are evenly applied throughout the stacks 14, 55. As used relative to the sheets 18, 38, 58, 78, the term “identical” may refer to being formed to include the same shape, size, thickness, and weight. Moreover, the term “substantial,” as used throughout the entire disclosure, may refer to any quantitative or qualitative representation that could permissibly vary without resulting in a change in the basic function to which it is related. For example, the term “substantially” may refer to any value within 5% of an indicated value. With regard to the similarity of the sheets in the stacks 14, 55, “substantially” identical sheets may be sheets in which the properties described above such as shape, size, etc. vary by a percentage value. As a non-limiting example, substantially identical sheets may be 90% identical, in which the size, thickness, and weight are within 90% of the same values of other sheets in the stacks 14, 55. The use of identical sheets can enable simplification of forming the sheets and fabricating the clutch device 10, as well as can provide simplicity in its design. The foregoing notwithstanding, a person skilled in the art will appreciate that using identical sheets is by no means necessary for the function of the clutch device 10. In fact, a person skilled in the art will understand that specifically tailored, non-identical sheets can possibly result in even better performance in certain usage scenarios.

A person skilled in the art will understand that the shapes, sizes, thicknesses, weights, and the like of some sheets 18, 38, 58, 78 in each plurality of sheets 16, 36, 56, 76 can deviate from the shapes of other sheets 18, 38, 58, 78 while still allowing the stacks 14, 55 to interlock with each other according to the working principles of the clutch device 10 described herein. Illustratively, the sheets 18, 38, 58, 78 may be formed of a thin material such as paper. A person skilled in the art will understand that nearly any thin film shape material that is not overly soft and/or overly sticky that will effectively engage and disengage each other can be utilized for the material of the sheets 18, 38, 58, 78, or more generally, for flexible members. As non-limiting examples, such materials may include plastic films, such as polyethylene terephthalate (PET) film and/or Polyimide film, and/or metal films, such as steel film, aluminum film, and/or copper film. Further, a person skilled in the art will understand that the sheets can include texturing, such as a roughened or patterned texture on their surfaces, to influence the interlocking and/or friction properties of the interleaved sheets.

In some embodiments, the pluralities of sheets 16, 36, 56, 76 can be constructed by interleaving two stacks of paper sheets 18, 38, 58, 78. The sheets 18, 38, 58, 78 can be rectangular pieces, for example, measuring about 75 mm×about 25 mm and can be cut from 0.1 mm thick copy paper (e.g., recycled copy paper, 8.5×11 inches, Staples) using a laser cutter (e.g., Fusion 40, Fusion Laser). To facilitate the assembly process, five holes 25, 45, 65, 85 can be laser cut on each layer, for example at a position about 12.5 mm from second terminal ends 23, 43, 63, 83. A person skilled in the art will understand that there is virtually no limitation on the size of the sheets 18, 38, 58, 78, although if the sheets are extremely small, on the order of micrometers, the stiction effect observed in MEMS devices, where significant Van der Waals adhesion occurs, may prevent the sheets from opening up to unlock. Conversely, if the sheets are too large, the bending force might not be sufficient to overcome their weight and allow them to open up to unlock. This balance depends, at least in part, on the weight and/or stiffness of the sheets used.

A person skilled in the art will understand that a variety of fabrication methods known in the art may be utilized to form the stacks 14, 55 described herein, and/or any other embodiments of stacks, layers, flexible members, etc. described, contemplated, or otherwise derivable from the present disclosures. As a non-limiting example, the pluralities of sheets 16, 36, 56, 76 can be formed via a stacking process in which each sheet is layered on an adjacent sheet until the final plurality or stack of sheets is formed. In some embodiments, the stacks 14, 55 can be formed via a similar stacking process, but instead by sequentially layering the sheets in an alternating format so as to form the interleaved arrangement while stacking (i.e., a first sheet from the first plurality of sheets 16 is layered, then a first sheet from the second plurality of sheets 36 is layered on top of said first sheet, and so on).

In some embodiments, the pluralities of sheets 16, 36, 56, 76 can be formed as interdigitated elements via additive manufacturing and/or any other 3D printing process. The types of printing devices used in conjunction with the same can provide desirable precision, and can allow for different regions of the stacks 14, 55 to be formed to have differing mechanical and/or structural properties. For example, certain regions, such as the overlapping regions, which is where the sheets 18, 38, 58, 78 engage and contact each other, can be formed differently than the non-overlapping regions so as to enhance or diminish certain properties of their engagement with each other. Printing devices used in conjunction with the same can have different regions along its printing area where the differently formed portions of the sheets are printed, thus providing a variety of properties across the surface being printed. Thus, the printing device itself can include different regions within the printer that provide different mechanical properties to a printed surface. A person skilled in the art will understand that other similar methods may be utilized to form the stacks 14, 55 as well.

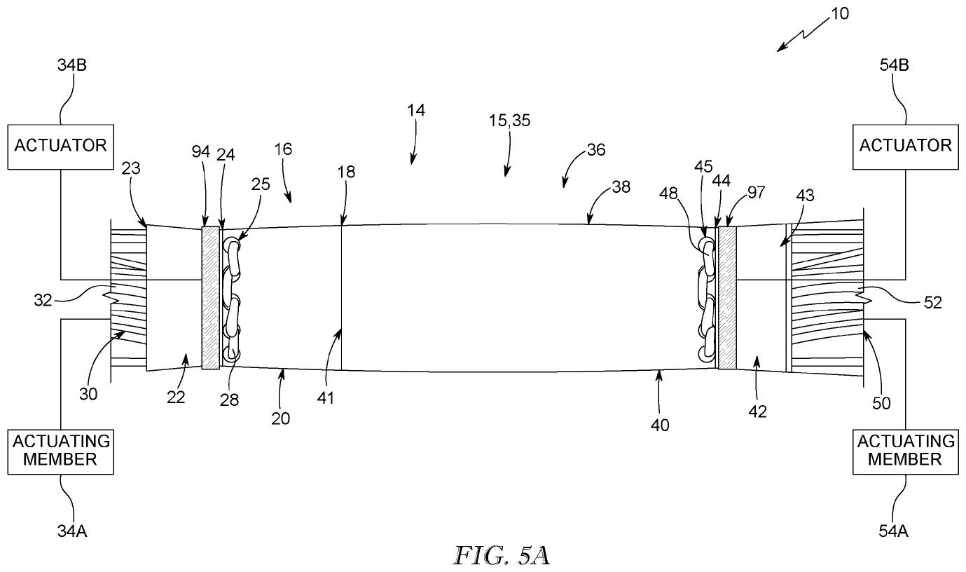

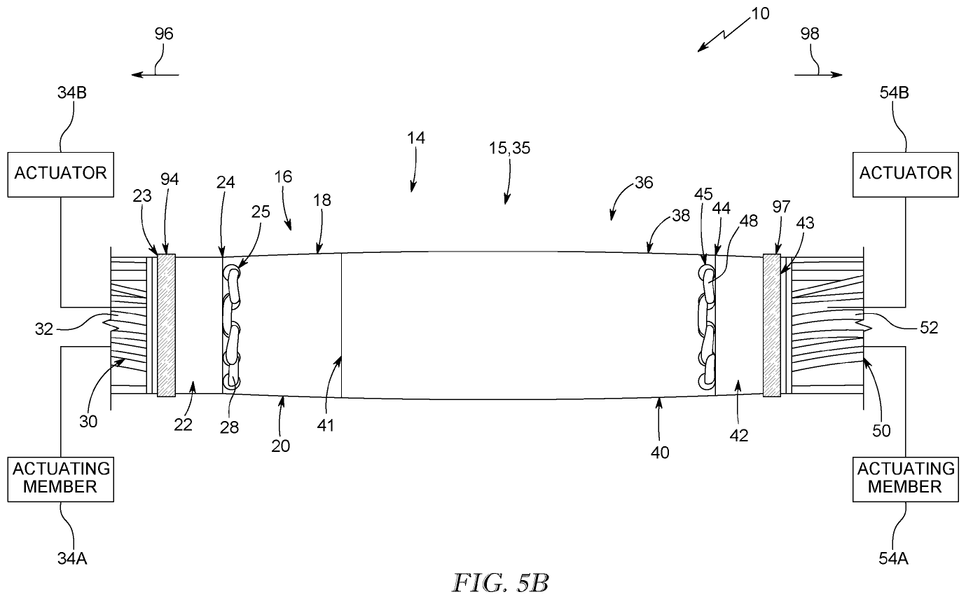

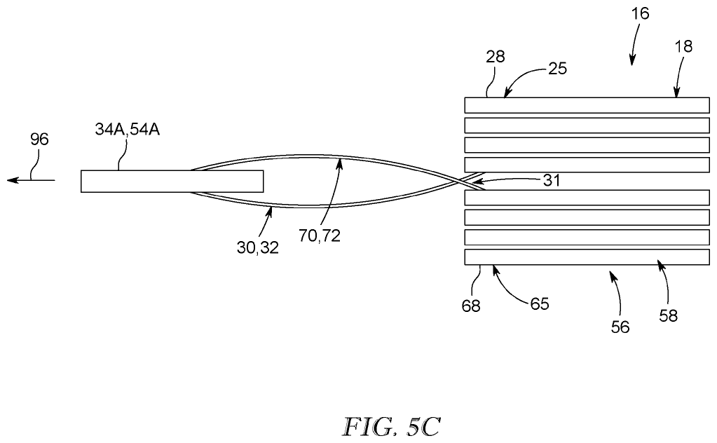

As can be seen in the top views shown in FIGS. 3A, 5A, and 5B, each sheet 18, 38, 58, 78 of the pluralities of sheets 16, 36, 56, 76 can include an elongated rectangular shape, although a person skilled in the art will understand that other shapes may be utilized based, at least in part, on aspects such as the design requirements of the clutch device 10 and/or its applications. Illustratively, the first and second pluralities of sheets 16, 36 can include the same number of sheets 18, 38, and the third and fourth pluralities of sheets 56, 76 can include the same number of sheets 58, 78. In some embodiments, the first, second, third, and fourth pluralities of sheets 16, 36, 56, 76 each include the same number of sheets 18, 38, 58, 78. As a non-limiting example, the first, second, third, and fourth pluralities of sheets 16, 36, 56, 76 can each include a number of sheets 18, 38, 58, 78 approximately in a range of about 10 sheets to about 100 sheets. In some embodiments, the first, second, third, and fourth pluralities of sheets 16, 36, 56, 76 can each include about 40 sheets. In some embodiments, the first, second, third, and fourth pluralities of sheets 16, 36, 56, 76 can each include about 60 sheets. In some embodiments, the first, second, third, and fourth pluralities of sheets 16, 36, 56, 76 can each include about 80 sheets. As will be described below, because the locking force of the clutch device 10 is at least partially based on the number of sheets in each plurality of sheets 16, 36, 56, 76, a person of skill in the art will understand that the number of sheets can be adjusted based, at least in part, on the design requirements of the clutch device and/or its applications.

The sheets 18, 38, 58, 78 of the stacks 14, 55 of the clutch device 10 are interleaved with each other, and thus overlap with each other, so as to form an overlapping region 15, 35, 75, 95 (also represented by L₀ in FIGS. 1 and 2) in which the sheets 18, 58 of the first stack 14 overlap with the sheets 38, 78 of the second stack 55, and a non-overlapping region 20, 40, 60, 80 in which the sheets 18, 58 of the first stack 14 do not overlap with the sheets 38, 78 of the second stack 55. In particular, each sheet 18, 38, 58, 78 includes a respective overlapping region 15, 35, 75, 95 and a non-overlapping region 20, 40, 60, 80. It is noted that the non-overlapping region 20, 40, 60, 80 is also partially represented by the separation distance (L_{f}) in FIGS. 1 and 2, which extends to the fold lines 24, 44, 64, 84, as also shown in FIG. 8A. Unlike the separation distance (L_{f}), the non-overlapping regions 20, 40, 60, 80 extend beyond the fold lines 24, 44, 64, 84 and include the distal end portions 22, 42, 62, 82.

As shown in FIGS. 1-3A and 4B, each sheet 18, 38, 58, 78 includes a first terminal end 21, 41, 61, 81 located at an inner side of the sheet 18, 38, 58, 78 (“inner” being a direction toward the center of the clutch device 10) and a second terminal end 23, 43, 63, 83 located opposite the first terminal end 21, 41, 61, 81 at an outer side of the sheet 18, 38, 58, 78 (“outer” being a direction away from the center of the clutch device 10). The overlapping region 15, 35, 75, 95 can be defined between first terminal ends 21, 61 of the first and third pluralities of sheets 16, 56 and first terminal ends 41, 81 of the second and fourth pluralities of sheets 36, 76. The non-overlapping regions 20, 40, 60, 80 of each sheet 18, 38, 58, 78 can be defined between the outer sides of the overlapping region 15, 35, 75, 95 where the opposing first terminal ends 21, 41, 61, 81 are located and the second terminal end 23, 43, 63, 83.

As will be described in greater detail below, the interleaved sheets 18, 38, 58, 78 of the pluralities of sheets 16, 36, 56, 76 can be arranged in such a way that the outermost sheets 18, 38, 58, 78 exert a small normal toward the middle of the stacks 14, 55, thus locking the stacks 14, 55 together (i.e. preventing the stacks 14, 55 from sliding away from each other or being separated entirely from each other). This force can be removed, for example, by rotating the pluralities of sheets 16, 36, 56, 76 in the directions shown by the arrows in FIG. 2, thus allowing the stacks 14, 55 to be unlocked. In one illustrative embodiment, as shown in FIGS. 3A-5B, rotation can be applied to the pluralities of sheets 16, 36, 56, 76 via rotation of distal end portions 22, 42, 62, 82 of the sheets 18, 38, 58, 78.

Specifically, as illustrated, the distal end portions 22, 42, 62, 82 can be bent outwardly at an angle 22A, 42A, 62A, 82A relative to the respective outer portion 18A, 38A, 58A, 78A of the sheet 18, 38, 58, 78 and relative to the central longitudinal axis 11 (angles 22A, 42A, 62A, 82A are shown relative to reference lines 11A, 11B, 11C, 11D in FIGS. 3A and 4A, which are substantially parallel to the central longitudinal axis 11). A person skilled in the art will understand that the angle 22A, 42A, 62A, 82A may be approximately in a range of about 0 degrees to about 90 degrees. As shown in FIGS. 3A, 4A, and 4B, and more clearly in 3B, 5A, and 5B, the distal end portion 22, 42, 62, 82 of each sheet 18, 38, 58, 78 is defined between the second terminal end 23, 43, 63, 83 and a fold line 24, 44, 64, 84 (also referred to herein as a “sewing point”) at which the sheet 18, 38, 58, 78 bends outwardly. In some embodiments, the distal end portions 22, 42, 62, 82 can be plastically bent outwards relative to the remainder of the sheet 18, 38, 58, 78 and relative to the middle of the stack (i.e. the central longitudinal axis of the clutch device 10).

As a non-limiting example, the length (L₀) of the overlapping region 15, 35, 75, 95 of each sheet 18, 38, 58, 78 can be approximately one-half of the total length (L_{T}) of the sheet 18, 38, 58, 78, as shown in FIG. 4B. A person skilled in the art will understand that this length can be adjusted based, at least in part, on the design requirements of the clutch device 10 and/or its applications. The non-overlapping region 20, 40, 60, 80 can occupy the other half of the total length (L_{T}) of the sheet 18, 38, 58, 78, and can include the area denoted by the separation distance (L_{f}) and the length of the distal end portions (L_{D}). The fold lines 24, 44, 64, 84 can divide the separation distance (L_{f}) and the length of the distal end portions (L_{D}), and can define a pivot axis about which the pluralities of sheets 16, 36, 56, 76 can be rotated. As will be described below, a person skilled in the art will understand that the separation distance (L_{f}), and thus the length of the distal end portions (L_{D}), can be adjusted based, at least in part, on the design requirements of the clutch device 10 and/or its applications. In at least some embodiments, the length of the distal end portions (L_{D}) is smaller than the separation distance (L_{f}). As a non-limiting example, the length of the distal end portion (L_{D}) is less than a quarter of the total length (L_{T}) of the sheet 18, 38, 58, 78. In some embodiments, each fold line 24, 44, 64, 84 is located closer to the second terminal end 23, 43, 63, 83 of the respective sheet 18, 38, 58, 78.

Each plurality of sheets 16, 36, 56, 76 can further include a tensioning assembly 30, 50, 70, 90 configured to pull the upper and lower pluralities of sheets (first and third pluralities of sheets 16, 56 and second and fourth pluralities of sheets 36, 76) toward each other and apply tension to the stacks 14, 55, also referred to as an external load on the stacks 14, 55. The external load may also be referred to as a lateral force. The lateral force combined with the geometry of the stacks 14, 55 can cause additional lateral forces to be applied and therefore lock the stacks 14, 55 together. The lateral force can also be applied via the bending of the distal end portions 22, 42, 62, 82, as described herein, or by other known means, such as twisting in embodiments in which the sheets 18, 38, 58, 78 are comprised of fibers, for example.

The tensioning assemblies 30, 70 can be configured to hold the first and third pluralities of sheets 16, 56 proximate to each other, and the tensioning assemblies 50, 90 can be configured to hold the second and fourth pluralities of sheets 36, 76 proximate to each other. The tensioning assemblies 30, 50, 70, 90 are also configured to compress the sheets 18, 38, 58, 78 of each of the pluralities of sheets 16, 36, 56, 76 together. As will be described below, this tension aids in amplifying the normal force exerted on the stacks 14, 55, thus increasing the locking force of the clutch device 10.

As can be seen in FIGS. 3A-5B, the tensioning assemblies 30, 50, 70, 90 each include a plurality of cords 32, 52, 72, 92 that are interwoven through the respective plurality of sheets 16, 36, 56, 76, as well as interwoven through each other. At or adjacent to the fold line 24, 44, 64, 84, each sheet 18, 38, 58, 78 can include a plurality of holes 25, 45, 65, 85 through which the plurality of cords 32, 52, 72, 92 extend. The holes 25, 45, 65, 85 can be evenly spaced apart in a line configuration adjacent to the fold line 24, 44, 64, 84. A person skilled in the art will understand that the cords 32, 52, 72, 92 can be any type of physical line or string configured to hold the pluralities of sheets 16, 36, 56, 76 in tension. As non-limiting examples, the cords 32, 52, 72, 92 can include string, rope, twine, wires, or the like. A person skilled in the art will understand that other means of securing the pluralities of sheets 16, 36, 56, 76 together can be utilized, with the pluralities of sheets 16, 36, 56, 76 typically being held in tension together at or near the fold lines 24, 44, 64, 84. Such alternative means can include, but are not limited to, rigid U-shaped support bars that hold the pluralities of sheets 16, 36, 56, 76 in the positions shown in FIGS. 1-5B while still allowing for rotation of the stacks 14, 55.

FIG. 5C shows a simplified schematic representation of how the cords 32, 52, 72, 92 can be interwoven through the pluralities of sheets 16, 36, 56, 76 and through themselves. Although only the tensioning assemblies 30, 70 of the first and third pluralities of sheets 16, 56 are shown, the same concept applies to the other tensioning assemblies 50, 90. As can be seen in FIG. 5C, the individual cords 32, 72 can be attached to a tensioning member 34A, 54A at one end. The cords 32, 72 also can be looped through the holes 25, 65 formed in the sheets 18, 58 and interwoven and/or sewn therethrough to form loops 28, 68 configured to pull the pluralities of sheets 16, 56 toward each other when tension is applied to the tensioning assemblies 30, 70. The cords 32, 72 can also be interwoven and/or sewn with each other and overlap at a location 31, as shown in FIG. 5C. A person skilled in the art will understand that the cords 32, 52, 72, 92 can be tightened at the holes 25, 45, 65, 85 to bring the sheets 18, 38, 58, 78 together, while the cords 32, 52, 72, 92 can also be tensed, for example due to being coupled to an external fixture that pulls on the cords 32, 52, 72, 92, thus bringing the sheets 18, 38, 58, 78 together.

In some embodiments, the stack can be sewn onto, for example, a 0.25 mm thick steel shim (McMaster Carr) (i.e. tensioning member 34A, 54A), a component later clamped to the test grips, through the holes 25, 45, 65, 85 cut with a waterjet cutter (MicroMAX, OMAX) using twine 32, 52, 72, 92 (Nylon twine, 0.046-inch diameter, McMaster-Carr), as shown in FIG. 3A. The paper stacks 14, 55 can be sewn to the steel shim (tensioning member 34A, 54A) in a twisting manner. As described above, the twine 32, 52, 72, 92 can initially traverse the top half of the paper stacks 14, 55 from top to bottom, then pass through the shim 34A, 54A from bottom to top, and finally be threaded through the bottom half of the paper stacks 14, 55 from top to bottom again, as depicted in FIG. 6A. Compared to the sewing method where both paper stacks 14, 55 and shim 34A, 54A are sewn from top to bottom, as shown in FIG. 6B, the twisting manner of sewing shown in FIG. 6A can ensure that the force exerted by the twine 32, 52, 72, 92 consistently pulls the two halves of the stacks 14, 55 together.

In this way, when the tensioning member 34A, 54A is pulled in a direction 96, the cords 32, 72 can pull the plurality of sheets 16, 56 toward each other under a tension force. This tension force can create a slight outward curvature in the pluralities of sheets 16, 36, 56, 76, as can be seen in FIG. 4A and schematically in FIG. 1. As will be described in detail below, due at least in part to this curvature, the non-overlapping regions 20, 40, 60, 80 of the sheets 18, 38, 58, 78 of the pluralities of sheets 16, 36, 56, 76 can be slightly inclined relative to the overlapping regions 15, 35, 75, 95 of the sheets 18, 38, 58, 78. In some embodiments, the surface of each overlapping regions 15, 35, 75, 95 of each sheet 18, 38, 58, 78 defines an overlapping region plane 15P, 35P, 75P, 95P (see FIG. 2) and at least a portion 18A, 38A, 58A, 78A of each non-overlapping region 20, 40, 60, 80 of the sheets 18, 38, 58, 78 each extend at a slight angle 16A, 36A, 56A, 76A relative to the overlapping region plane 15P, 35P.

For ease of illustration and understanding, as shown in FIGS. 1 and 2, the overlapping region plane 15P, 35P, 75P, 95P is substantially parallel with the central longitudinal axis 11 such that the angle 16A, 36A, 56A, 76A formed between the overlapping region plane 15P, 35P, 75P, 95P and the non-overlapping region 20, 40, 60, 80 is also formed between the central longitudinal axis 11 and the non-overlapping region 20, 40, 60, 80. A person skilled in the art will understand that the overlapping region plane 15P, 35P, 75P, 95P need not be substantially parallel with the central longitudinal axis 11, so long as there is an angle 16A, 36A, 56A, 76A formed between the overlapping region plane 15P, 35P, 75P, 95P and the non-overlapping region 20, 40, 60, 80.

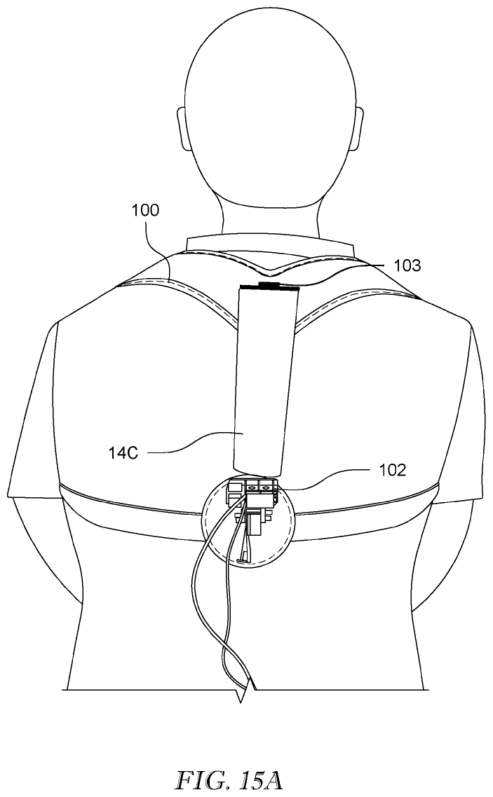

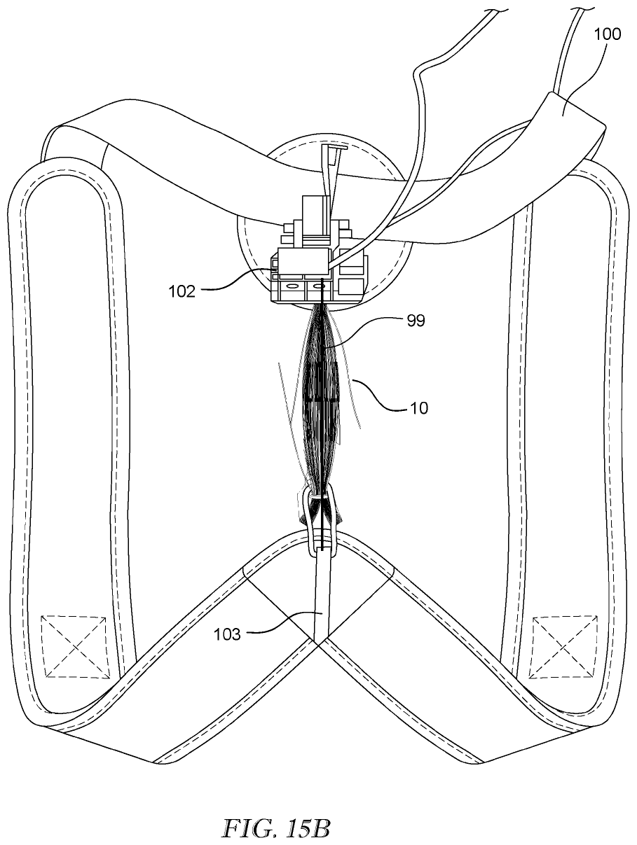

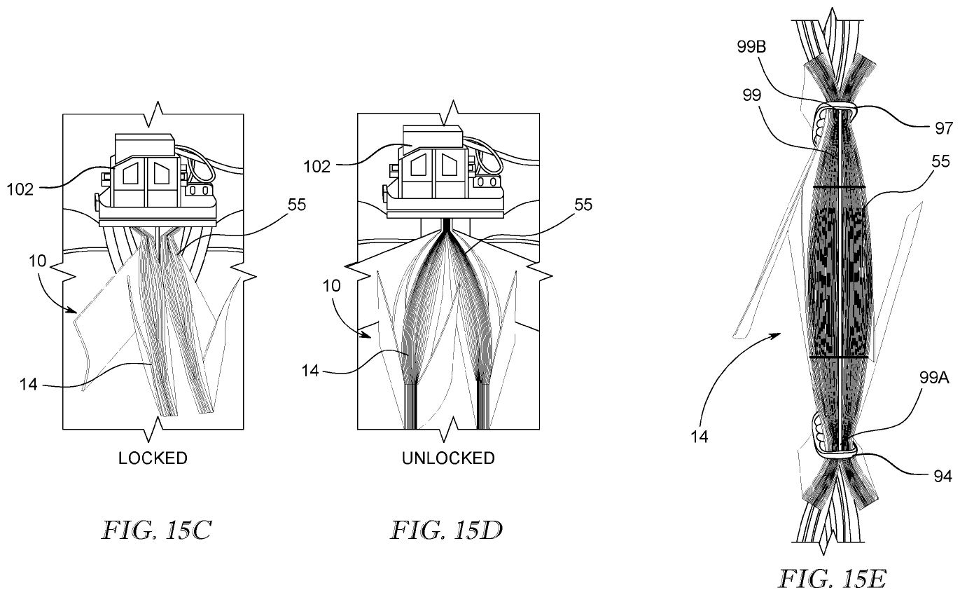

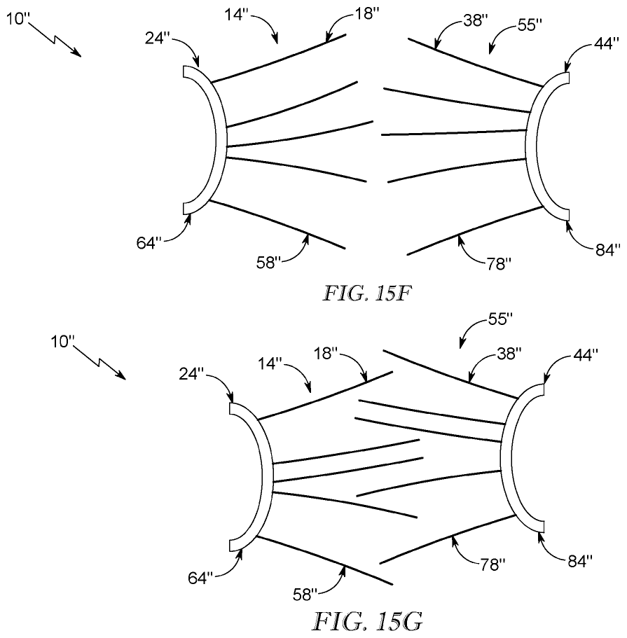

This incline of the non-overlapping regions 20, 40, 60, 80 relative to the corresponding overlapping regions 15, 35, 75, 95 can cause the non-overlapping regions 20, 40, 60, 80 to exert a normal force onto the overlapping regions 15, 35, 75, 95. As will be described in detail below with regard to the working principles of the device 10 (“Working Principles of the Clutch Device” section), the normal force imparts a frictional force between the sheets 18, 38, 58, 78, this friction force preventing the sheets 18, 38, 58, 78 from slipping and being released. The normal and friction forces thus lock the stacks 14, 55 together. Increasing the tension force created by the tensioning assemblies 30, 50, 70, 90 can amplify this normal force and thus the friction force. In practical usage scenarios, the tensioning member 34A, 54A can be directly attached to two bodies so that relative motion between the two bodies is prevented due to the locking properties of the clutch device 10 (e.g. FIGS. 15A-15E).

The normal force can be removed to in turn allow the clutch device 10 to be unlocked, for example, by rotating the distal end portions 22, 42, 62, 82 of the sheets 18, 38, 58, 78 to rotate the sheets 16, 36, 56, 76, as shown progressively in FIGS. 4A and 4B. For example, as shown schematically in FIG. 1, outer portions 18A, 38A, 58A, 78A of each non-overlapping region 20, 40, 60, 80 of the sheets 18, 38, 58, 78 located adjacent to the fold lines 24, 44, 64, 84 each extend at a slight angle 16A, 36A, 56A, 76A relative to the overlapping region plane 15P, 35P, 75P, 95P and to the central longitudinal axis 11. Again, this angle 16A, 36A, 56A, 76A is what can cause the non-overlapping regions 20, 40, 60, 80 to exert the normal force onto the overlapping regions 15, 35, 75, 95.

FIG. 2 schematically shows the pluralities of sheets 16, 36, 56, 76 after having been rotated by an angle θ about the fold line 24, 44, 64, 84, which is effectively a pivot axis of the respective plurality of sheets 16, 36, 56, 76. The rotation angle θ is also represented by the difference in the initial angle 22A, 42A, 62A, 82A of the bend in the distal end portions 22, 42, 62, 82 and the resulting angle 22B, 42B, 62B, 82B of the distal end portions 22, 42, 62, 82 after rotation of the pluralities of sheets 16, 36, 56, 76, as shown in FIGS. 4A and 4B. As can be seen in FIG. 2, after the pluralities of sheets 16, 36, 56, 76 are rotated, the angles of the outer portions 18A, 38A, 58A, 78A of the non-overlapping regions 20, 40, 60, 80 of the sheets 18, 38, 58, 78 relative to the overlapping region plane 15P, 35P, 75P, 95P and to the central longitudinal axis 11 are increased to the angles 16B, 36B, 56B, 76B. This increase in the angle moves the stacks 14, 55 into the unlocked state. As will be described in detail below, the magnitude of the difference between the initial angle 16A, 36A, 56A, 76A and the angle 16B, 36B, 56B, 76B after rotation can have an effect on how great of a normal force can be overcome by the rotation.

A person skilled in the art will understand that the initial angle 16A, 36A, 56A, 76A and the angle 16B, 36B, 56B, 76B after rotation need not be identical for each successive sheet 18, 38, 58, 78 in the respective plurality of sheets 16, 36, 56, 76, and can vary between centermost and outermost sheets 18, 38, 58, 78. For example, the centermost sheets 18, 38, 58, 78 (i.e. those sheets located closest to the central longitudinal axis 11 of the clutch device 10) and the outermost sheets 18, 38, 58, 78 (i.e. those sheets located furthest from the central longitudinal axis 11 of the clutch device 10) may possess the same or different initial angle 16A, 36A, 56A, 76A. A person skilled in the art will understand that the terms “centermost” and “outermost” encompass the sheets that are generally located in these areas relative to the longitudinal axis 11, such as, for example, the outer half of the sheets being the outermost sheets 18, 38, 58, 78 and the inner half of the sheets being the centermost sheets 18, 38, 58, 78. In some embodiments, the rotation of the sheets 18, 38, 58, 78 may be referred to as the sheets “bending outwardly” (i.e. in a direction away from the central longitudinal axis 11 such that the concavity of the sheet 18, 38, 58, 78 relative to the axis 11 increases, as shown in FIG. 2 as compared to FIG. 1). As will be described in detail below, the rotation of the stacks 14, 55 (i.e. the “bending outwardly” movement) can contribute to the creation of the normal and friction forces that retain the clutch device 10 in the locked arrangement.

Rotation of the distal end portions 22, 42, 62, 82 of the sheets 18, 38, 58, 78 can be caused, at least in some embodiments, by clamps 94, 97 arranged on the distal end portions 22, 42, 62, 82 of the clutch device 10, as shown in FIGS. 4A-5B. A first clamp 94 can wrap around the distal end portions 22, 62 and can be configured to hold the first and third pluralities of sheets 16, 56 in a tensioned position. Similarly, a second clamp 97 can wrap around the distal end portions 42, 82 and can be configured to hold the second and fourth pluralities of sheets 36, 76 in a tensioned position. A person skilled in the art will understand that the clamps 94, 97 are not required to hold the pluralities of sheets 16, 36, 56, 76 in the tensioned positions, but can assist in doing so, as the tension is generally created via the tensioning assemblies 30, 50, 70, 90. Each clamp 94, 97 can be configured, for example, to slide along the respective distal end portion 22, 42, 62, 82 in directions 96, 98, as shown in FIGS. 4A-5B. As a result, when the clamp 94, 97 is slid outwardly toward the second terminal ends 23, 43, 63, 83, the clamp 94, 97 pulls the distal end portions 22, 42, 62, 82 closer together, thus causing the rotation of the pluralities of sheets 16, 36, 56, 76 to unlock the clutch device 10. In the unlocked state, the first and second stacks 14, 55 are able to be entirely separated from each other.

In some embodiments, the clamps 94, 97 can be formed as rectangular, solid clamps formed to substantially match the thickness of the respective pluralities of sheets 16, 56, 36, 76 and hold the pluralities of sheets 16, 56, 36, 76 in tension. Illustratively, the clamps 94, 97 can be formed as rubber bands that securely wrap around the respective pluralities of sheets 16, 56, 36, 76. In some embodiments, the clamps 94, 97 may be operably connected to external actuators 34B, 54B that can be configured to slide the clamps 94, 97 along the distal end portions 22, 42, 62, 82. A person skilled in the art will understand that any material capable of performing the clamping function may be utilized, and that such materials may allow for sliding of the clamps 94, 97. In some embodiments, the clamp 94, 97 may be opened, subsequently moved from the initial position and toward the second terminal ends 23, 43, 63, 83, and then closed to create the rotation described above.

As shown in FIGS. 6A and 6B, the clutch device 10′ may be arranged in a “half-stack” arrangement as opposed to a “full-stack” arrangement, as described above. The “half-stack” arrangement clutch device 10′ is designed to operate in the same manner and operate under the same working principles as the “full-stack” arrangement. For example, as can be seen in FIGS. 6A and 6B, the clutch device 10′ includes the same components as the clutch device 10 except only the upper half of the clutch device 10 is utilized. For example, instead of four pluralities of sheets 16, 36, 56, 76 comprising the stacks 14, 55, only two pluralities of sheets 16′, 36′ are included, with each including the same configuration of sheets 18′, 38′ and distal end portions 22′, 42′. In embodiments including this “half-stack” design, the clutch device 10′ is unlocked via rotation of the distal end portions 22′, 42′ to cause removal of the normal force imparted by the non-overlapping regions 20′, 40′ on the overlapping regions 15′, 35′.

### Working Principles of Disclosed Clutch Devices

The clutch devices 10′, 10″, 210, 310 of the present disclosure operate according to the following described working principles. These principles relate to the geometrically amplified friction of interleaved layers or sheets, such as the interleaved layers or sheets 18, 38, 58, 78 of the pluralities of sheets 16, 36, 56, 76. When the sheets 18, 38, 58, 78 of the pluralities of sheets 16, 36, 56, 76 of the two stacks 14, 55 are interleaved, each sheet 18, 38, 58, 78 follows a diagonal, inclined path (angle 16A, 36A, 56A, 76A) in the non-overlapping region 20, 40, 60, 80 (also shown as L_{f} in FIGS. 1 and 2) from the sewing point (fold line 24, 44, 64, 84) until it reaches the overlapping region 15, 35, 75, 95 (also shown as L₀ in FIGS. 1 and 2). Because of this geometric accommodation, when the clutch device 10′, 10″, 210, 310, is loaded in tension, the force exerted by the non-overlapping region 20, 40, 60, 80 onto the overlapping region 15, 35, 75, 95 not only balances a frictional force between the sheets 18, 38, 58, 78, but also applies a normal force toward the middle of the stack (centrally toward a center of the central longitudinal axis 11 shown in FIGS. 1 and 2). As a result, the normal force is amplified toward the middle of the stack. This amplification effect extends to the frictional strength of the sheets 18, 38, 58, 78, in accordance with Amontons-Coulomb laws.

To ensure structural integrity and durability, the stacks 14, 55 can be sewn together using, by way of non-limiting example, twine (i.e. cords 32, 52, 72, 92) that passes through the stacks 14, 55, as shown a middle of the stacks 14, 55, though it can pass through one or more other locations. In real-world applications, the twine 32, 52, 72, 92 that sews the stacks 14, 55 together can also be sewn to the components that require the clutch to provide switchable engagement (for example, the two ends of the posture corrector shown in FIGS. 15A-15E). The sewing technique provides benefits such as its superior ability to withstand the forces exerted on the clutch device 10′, 10″, 210, 310 when used in real-word scenarios. A person skilled in the art will understand that other methods of holding the stacks 14, 55 together under tension can be used, such as, but not-limited to, glueing the sheets to a fixture.

In conjunction with creating the various clutch embodiments disclosed herein, a plurality of experiments were performed to help design the same. For these experiments, clutches with different numbers of layers, 2M=40, 60, 80, in each stack were fabricated, resulting in respective clutch thicknesses of about 8 mm, about 12 mm, and about 16 mm, respectively. The tensile properties of the clutches were tested in a standard universal testing machine (Instron 5567) equipped with a 5 kN load cell. To mount the clutches to the testing machine, the twine 32, 52, 72, 92 that sews each stack was sewn to a steel shim 34A, 54A, and the two shims 34A, 54A were clamped onto the testing machine using wedge action grips. Tensile tests were performed under displacement control at a rate Δd=10 mm/min, unless otherwise specified. Both force and displacement data were recorded throughout the testing process. The clutch device 10 was locked in its as-fabricated configuration, as shown in FIG. 1, which permits direct measurement of its tensile properties. The strength in the unlocked configuration was measured by clamping the distal ends of the clutch device 10 together (Plastic clothespins, Amazon), like the configuration depicted in FIG. 2.

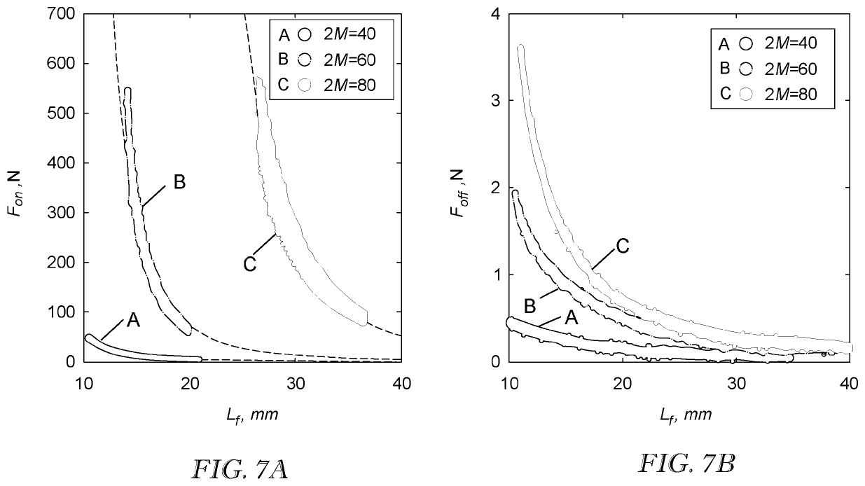

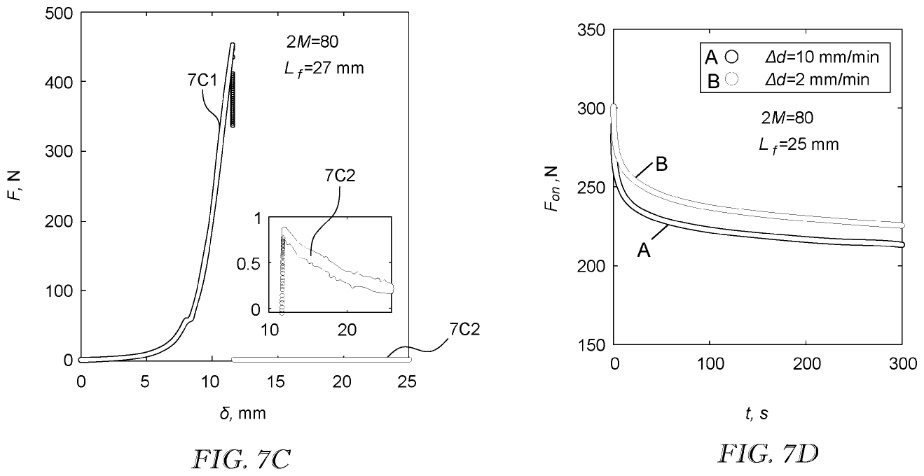

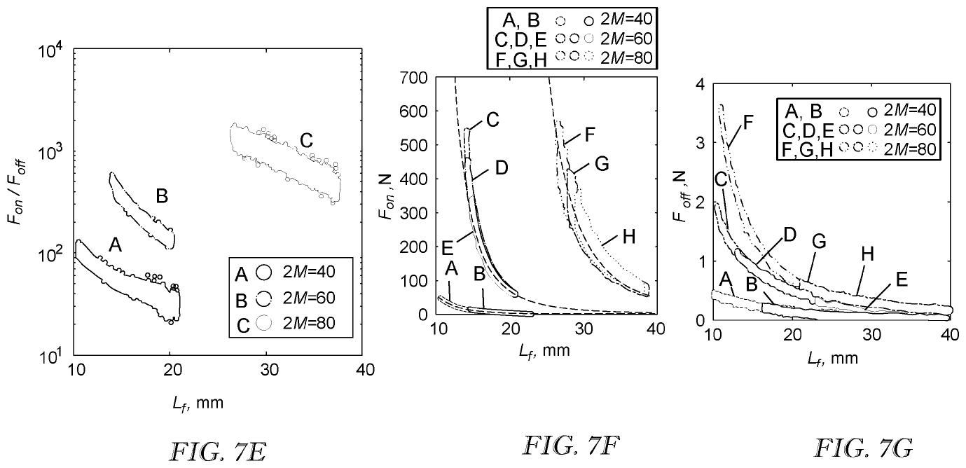

One of the primary functions of a clutch is to sustain a tensile force without slipping. For the clutch device 10, FIG. 7A illustrates the measured frictional strength (Fₒₙ) in the locked state, which represents a maximum tensile force the clutch device 10 can sustain without slipping, versus the separation distance (L_{f}) (see FIG. 8A), for exemplary clutch devices 10 with different numbers of sheets 18, 38, 58, 78 (2M=40, 60, 80) in each stack 14, 55. The measured frictional strength (Fₒₙ) in the locked state serves as an upper limit, and in real-world scenarios, the clutch device 10 may be configured to operate below this limit to prevent the risk of slipping. The separation distance (L_{f}) is measured from the sewing point (fold line 24, 44, 64, 84) to the overlapping region 15, 35, 75, 95. The total distance between the sewing points (fold line 24, 44, 64, 84) of the two stacks is L+L_{f}, where L is the length of the layer from the sewing point (fold line 24, 44, 64, 84) to the first terminal ends 21, 41, 61, 81 (62.5 mm for the clutches tested). There exists an exponential increase in the frictional strength (Fₒₙ) as the separation distance (L_{f}) decreases or as the layer number (2M) increases. Therefore, the strength (Fₒₙ) of the clutch device 10 for a given separation distance (L_{f}) can be tailored, for example, by adjusting the number of sheets 18, 38, 58, 78. Exemplary clutch devices 10 with 2M=60 or 80 layers achieve a maximum force of approximately 550 N, as shown in FIG. 7A. The observed limitation to a maximum force of approximately 550 N in the experiments is attributed to the twine 32, 52, 72, 92 being severed by a steel shim at loads higher than 550 N.

The ability of the clutch device 10 to maintain a given load at a specific displacement in the locked state (or vice versa, to hold a certain displacement with a particular load) also can be influenced by factors such as the time-dependent properties of the materials used in its construction. While the static frictional strengths (Fₒₙ) of the interfaces of the sheets 18, 38, 58, 78 can be expected to increase slightly during holding, as the static friction coefficient typically rises with dwell time, effectively preventing sliding during the holding period, the time-dependent modulus of the materials can have a contrasting impact on performance of the clutch device 10. The clutch device 10 can exhibit typical stress relaxation behavior when loaded to a specific force and then held, as depicted in FIG. 7D, due to the viscoelastic nature of paper (used as the material for the sheets 18, 38, 58, 78 in this exemplary embodiment). As such, when optimal performance of the clutch device 10 during prolonged holding is desired, it can be helpful to select materials with little or no creep at the required loads.

In addition to high frictional strength in the locked state, a switchable clutch can also benefit from low frictional strength in the unlocked state to minimize resistance to motion. To evaluate this aspect, the frictional strength of the clutch device 10 at the unlocked state (F_{off}), which is the resistive tensile force the clutch device 10 exhibits during sliding in the unlocked state, was measured by clamping the distal end portions 22, 42, 62, 82 using clips or clamps, such as the clamps 94, 97 described above. FIG. 7B shows the measured frictional strength (F_{off}) in the unlocked state as a function of the separation distance (L_{f}) for clutch devices 10 with different layer numbers (2M=40, 60, 80). The values of the friction strength in the unlocked state (F_{off}) are on the order of a few N. Reducing the separation distance (L_{f}) and increasing the sheet or layer number both contribute to increased frictional strength in the unlocked state.

The switchability of the clutch device 10 can be evaluated, for example, by dividing the locked state strength (Fₒₙ) by the unlocked state strength (F_{off}) at the same separation distance (L_{f}), utilizing the experimental data from FIGS. 7A and 7B. The results, depicted in FIG. 7E indicate higher switchability for smaller separation distances (L_{f}) and a greater number of sheets or layers.

In some embodiments, the frictional force can exhibit fluctuations due, at least in part, to stick-slip behavior. However, the magnitude of these fluctuations can be relatively small compared to the overall magnitude of the force. Additionally, results indicate that the performance of the clutch device 10 remains substantially consistent regardless of the initial separation distance between the layers, as shown in FIGS. 7F and 7G. Therefore, for the purpose of the analysis disclosed herein, the effects of stick-slip behavior and the difference between static and kinetic friction can be neglected.

Another important characteristic of a clutch is the ability to unlock while under load, which distinguishes it from classic locking devices such as latches and ratchets that typically cannot unlock when subjected to a load. To ascertain whether the clutch devices disclosed herein, such as the clutch device 10, possess this capability, a tensile test was conducted using a clutch with 2M=80 layers and an initial separation distance of L_{f}=27 mm. During the test, the clutch device 10 was pulled until the sliding was observed, as indicated by the first curve 7C1 in FIG. 7C. At this point, the distal end portions 22, 42, 62, 82 of the clutch device 10 were rotated to the unlocked state before continuing with the pulling motion. Subsequently, the test continued, and the force vs. displacement relationship was recorded, resulting in the second curve 7C2 shown in FIG. 7C. It is evident from FIG. 7C that the clutch device 10 successfully unlocks while under load. This observation confirms the ability of the clutch device 10 to disengage and transition to the unlocked state even when subjected to external forces, which can be helpful for practical use.

To gain a deeper understanding of mechanical behavior of the clutch, an analytical model was developed. The model focuses on the interleaved stacks 14, 55 of sheets 18, 38, 58, 78, considering only half of the stacks 14, 55 (i.e. first and second pluralities of sheets 16, 36) with M layers on each side due to symmetry. For the purposes of this model, the sheets 18, 38, referred to as layers in much of the following discussion, are indexed from the centermost layer n=1 to the outermost layer n=M (see FIG. 1). As illustrated in FIG. 8A, the deflection (hₙ) of the nth layer in the non-overlapping region 20, 40 is nε, where ε represents the layer thickness. In the analysis, the deflections (hₙ) of all layers are small compared to the separation distance (L_{f}). This allows application of a small angle approximation to the deflection angle ξₙ (i.e., angles 16A, 36A), which can be approximated as ξₙ≈hₙ/L_{f}. By employing this approximation, a first-order model for the behavior of the clutch device 10 can be derived.

Due to this angle ξₙ, the horizontal traction Tₙ exerted on layer n results in a normal force Tₙhₙ/L_{f} directed towards the centermost layer n=1 of the stacks 14, 55. Additionally, in contrast to previous analytical frameworks that neglect the bending stiffness of the layer, the disclosed model considers the bending of the layer n, which generates an additional normal force, fₙ. Incorporating this bending force, fₙ, can be helpful for understanding the working principle of the clutch device 10. The bending force, fₙ, can be predicted by the Euler-Bernoulli beam theory, assuming small deflection angles ξₙ. Accounting for the interleaved assembly having two symmetrical stacks 14, 55, the normal contact force in the overlapping region 15, 35, 75, 95 experienced by adjacent layers or sheets 18, 38, 58, 78 (with each layer having two opposing contact interfaces both subject to a normal force) differs by 4 (Tₙhₙ/L_{f}+fₙ). According to Amontons-Coulomb laws, the following governing equation needs to be satisfied at the onset of sliding:

$${T}_{n - 1} - {T}_{n} \approx 4 μ ( \frac{{h}_{n}}{{L}_{f}} {T}_{n} + {f}_{n} ) , \qquad ( 1 )$$

where μ is the friction coefficient.

In the locked state, as shown in FIG. 8A, the bending force fₙ points towards the centermost layer n=1 of the stacks 14, 55. This bending force can be calculated using the equation fₙ=12 Dnε/L_{f}³, where D=Ewε³/12 is the flexural rigidity of the layer and E is the modulus (E=3 GPa), w is the width (w=25 mm), and ε is the layer thickness (ε=0.1 mm) used in the disclosed studies. Considering that M>>1, a continuous approximation can be adopted by introducing the parameter z=n/M and replacing Tₙ with T(z). To facilitate the analysis, two dimensionless parameters: α=2μεM²/L_{f} and β=48 μDεM²/L_{f}³ can be defined. By incorporating these dimensionless parameters, a continuous description of Equation (1) for the locked state can be derived, expressed as:

$${T}^{′} ( z ) + 2 α zT ( z ) + β z = 0. \qquad ( 2 )$$

The boundary condition for Equation 2 is T(1)=0 (i.e., the traction at the outermost layer n=M is negligible). By solving Equation 2 with this boundary condition, the following expression for T(z) can be obtained:

$$T ( z ) = \frac{β}{2 α} exp [ α ( 1 - {z}^{2} ) ] - \frac{β}{2 α} , \qquad ( 3 )$$

where exp( ) is the exponential function. Using Equation 3, the total force in the locked state can be calculated, denoted as Fₒₙ:

$${F}_{o n} = 2 M {\int }_{0}^{1} T ( z ) d z = 2 M [ \frac{β}{2 α} \sqrt{\frac{π}{4 α}} exp ( α ) erf ( \sqrt{α} ) - \frac{β}{2 α} ] . \qquad ( 4 )$$

The distribution of horizontal traction (Tₙ) within the stacks 14, 55, calculated using Equation 3, is illustrated in FIG. 8B. Tₙ increases as the layer index n decreases, indicating that the traction becomes larger as a layer gets closer to the middle of the stack (n=1). Furthermore, for a given layer index n, Tₙ increases with a larger total layer number (2M). In accordance with Equation 4, the total frictional strength of the stacks 14, 55 in the locked state (Fₒₙ) exhibits an exponential dependence on the parameter α, which scales with M² and 1/L_{f}. Consequently, increasing the number of layers or reducing the separation distance leads to a significant enhancement in the total frictional strength. Note that the overall thickness of the interleaved assembly is 4εM, while α is proportional to εM². This suggests that by keeping the thickness of the assembly unchanged (4εM), the Fₒₙ can be increased by using thinner layers to increase the number of layers.

It is noted that Equations 3 and 4 assume a constant friction coefficient (u), which is not strictly accurate in reality as μ may vary, for example, with the normal pressure. However, assuming a constant μ and treating it as a fitting parameter is a reasonable simplification for this assembly of the pluralities of sheets 16, 36. In line with this approach, the dashed lines in FIG. 7A represent the modeling results based on Equation 4, with u adjusted as a fitting parameter (μ=0.2 for 2M=40, μ=0.3 for 2M=60, and μ=0.4 for 2M=60). It is also noted that the value of β in Equation 4 is based on fₙ of the perfectly confined case:

$$( {f}_{n} = \frac{12 D {h}_{n}}{{L}_{f}^{3}} ) , \qquad ( 5 )$$

while in reality, the confinement is expected to be less for fewer layers, leading to a smaller fₙ close to:

$$( {f}_{n} = \frac{3 D {h}_{n}}{{L}_{f}^{3}} ) . \qquad ( 6 )$$

This reduction in fₙ (thus β) is also accounted for in the value of the fitting parameter μ here.

To unlock the clutch device 10, a rotation with angle θ can be applied to the distal end portions 22, 42, 62, 82 of the sheets 18, 38, 58, 78, as described above, resulting in a modified bending force fₙ=12 Dnε/L_{f}³−6 Dθ/L_{f}². This rotation θ introduces a force component −6 Dθ/L_{f}² that counteracts the self-amplified normal force Tₙhₙ/L_{f} and the bending force component 12 Dnε/L_{f}³ present in the locked state. To analyze this behavior, the same continuous approximation as used for the locked state can be adopted, and an additional dimensionless parameter γ=24 μDMθ/L_{f}² can be adopted as well. By incorporating these parameters, a continuous description of Equation 1 for the unlocked state can be derived, expressed as:

$${T}^{′} ( z ) + 2 α zT ( z ) + β z - γ = 0. \qquad ( 7 )$$

As the rotation angle θ increases, the force component −6 Dθ/L_{f}² also increases. Eventually, this force component can dominate over other normal force components (Tₙhₙ/L_{f}+12 Dnε/L_{f}³) and reverse the direction of the total normal force (Tₙhₙ/L_{f}+fₙ), causing it to point towards the outermost layer of the stack, as also shown in FIG. 8A. This critical transition occurs at θ=θ_{c} when T(0)=0 (i.e., the centermost layer n=1 becomes traction free).

For rotation angles θ≤θ_{c}, where Tₙhₙ/L_{f}+fₙ still points towards the centermost layer n=1, the boundary condition remains as T(1)=0. By solving Equation 7 with this boundary condition, the following expression for the traction T(z) is obtained:

$$T ( z ) = \frac{β}{2 α} exp ( α - α {z}^{2} ) - \frac{β}{2 α} - γ \sqrt{\frac{π}{4 α}} exp ( - α {z}^{2} ) [ erfi ( \sqrt{α} ) - erfi ( \sqrt{α} z ) ] , \qquad ( 8 )$$

where erfi( ) is an imaginary error function. The total force in the unlocked state, denoted as F_{off}, is given by:

$${F}_{off} = {F}_{o n} + 2 M [ - \frac{π γ}{4 α} erf ( \sqrt{α} ) erfi ( \sqrt{α} ) + \frac{γ}{2} {}_{2} {F}_{2} ( 1 , 1 ; 1.5 , 2 ; - α ) ] , \qquad ( 9 )$$

where Fₒₙ is given by Equation 4, erfi( ) is the error function, and ₂F₂( ) is a generalized hypergeometric function.

In FIG. 8C, the distribution of horizontal traction (Tₙ) within the stacks 14, 55 for various end rotation angles (θ) is shown when θ≤θ_{c}, indicating that the normal force (Tₙhₙ/L_{f}+fₙ) still points towards the centermost layer n=1. The plot clearly illustrates that as the applied rotation angle θ increases, the horizontal traction Tₙ decreases for all layers. This reduction in Tₙ occurs at least because the force component −6 Dθ/L_{f}² becomes larger and counteracts a larger portion of the force components Tₙhₙ/L_{f}+12 Dnε/L_{f}³. At the critical rotation angle θ=θ_{c}=0.22, the horizontal traction at the centermost layer becomes zero (i.e., T(0)=0). The value of θ_{c} depends, at least in part, on the layer number (M) and/or separation distance (L_{f}), and FIG. 8F provides a visual representation of θ_{c} for different M and L_{f} values.

If θ is further increased beyond θ_{c}, the direction of the total normal force (Tₙhₙ/L_{f}+fₙ) will reverse, pointing towards the outermost layer n=M. This reversal of the normal force can cause the stacks 14, 55 to fan outward, as depicted in FIG. 4B. In some embodiments, this fanning can be constrained by a cover 14C, as shown schematically in FIG. 8I. If the cover 14C is configured to have no gap (shown as distance δ) between itself and the overlapping region 15, 35, 75, 95 of the stacks 14, 55, assuming the cover 14C is rigid, the interleaved stacks 14, 55 can preserve the closely packed configuration observed in the unlocked state (see FIGS. 8A and 8I). In this scenario, further increasing θ beyond θ_{c} can increase the contact force between the outermost layer n=M and the cover 14C. Consequently, Tₙ can increase for all layers, as depicted in FIG. 8K.

However, in the presence of a gap of distance δ between the cover 14C and the pluralities of sheets 16, 36, as illustrated in FIG. 8I, the confinement can be loosened, resulting in a reduction of the contact force between the outermost layer n=M and the cover 14C, along with a decrease in Tₙ as δ increases FIG. 8J. As δ progresses to a critical value (δ_{c}), the contact force between the outermost layer n=M and the cover 14C diminishes, resembling a scenario where the cove 14C is exceedingly flexible, offering no resistance to the interleaved stacks 14, 55, or essentially mirroring the case with no cover. FIG. 8K provides a summary of the calculated critical gap distance (δ_{c}) for various layer numbers (M) and end rotation angles (θ), revealing that only a few millimeters of critical gap may be necessary. To estimate the frictional strength in the absence of effective confinement, the situation where the gap distance δ can be consistently maintained at the critical value δ_{c} can be analyzed. FIG. 8L illustrates that an increase in e can further reduction in the Tₙ within the stack.

The strength of the clutch device 10 in the unlocked state, F_{off}, can then be calculated by summing Tₙ of each layer. As represented by FIG. 8D, F_{off} as a function of the rotation angle θ, can be calculated using Equation 9 (above), Equation 10 (below), and Equation 11 (below):

$${F}_{off} = 2 M [ \frac{β}{2 α} \sqrt{\frac{π}{4 α}} erf ( \sqrt{α} ) - \frac{β}{2 α} + \frac{γ}{2} {}_{2} {F}_{2} ( 1 , 1 ; 1.5 , 2 ; - α ) ] , \qquad ( 10 )$$

$${F}_{off} = \frac{1}{16 {α}^{3}} ( \begin{matrix}{λ}^{2} ( λ β - 2 αρ ) {}_{2} {F}_{2} ( 1 , 1 ; \frac{3}{2} , 2 ; \frac{{λ}^{2}}{4 α} ) + \\ \begin{matrix}{( 2 α + λ )}^{2} ( 2 α ρ - λβ ) {}_{2} {F}_{2} \\ ( 1 , 1 ; \frac{3}{2} , 2 ; \frac{{λ}^{2}}{4 α} + λ + α ) - 4 \sqrt{π} {α}^{3 / 2} β {e}^{\frac{{λ}^{2}}{4 α}} erf ( \frac{λ}{2 \sqrt{α}} )\end{matrix} - \\ \begin{matrix}2 α ( erf ( \frac{2 α + λ}{2 \sqrt{α}} ) ( π ( erfi ( \frac{λ}{2 \sqrt{α}} ) - \\ erfi ( \frac{2 α + λ}{2 \sqrt{α}} ) ) ( λ β - 2 α ρ ) - 2 \sqrt{π} \sqrt{α} β {e}^{\frac{{λ}^{2}}{4 a}} ) + 4 α β )\end{matrix}\end{matrix} ) . \qquad ( 11 )$$

Solid lines depict the results for θ≤θ_{c}, whereas dashed lines represent the results for θ>θ_{c} under the condition of the stacks 14, 55 being closely confined by the cover 14C (δ=0). Additionally, dash-dotted lines represent results for θ>θ_{c} under the condition of no effective confinement (T(1)=0). For θ≤θ_{c}, F_{off} decreases with increasing θ, enabling the transition of the clutch device 10 into the unlocked state. However, for θ>θ_{c}, F_{off} experiences a subsequent increase as the rotation angle θ further rises under the condition of tight confinement. Meanwhile, under the scenario of no effective confinement, F_{off} continues to decrease. The analysis of the two confinement cases underscores the benefit of maintaining a sufficient gap between the cover 14C and the stacks 14, 55, a distance predicted to be only a few millimeters.

The analysis presented in FIGS. 8C and 8D and the accompanying discussion provide insights into the unlocking mechanism and sheds light on the influence of the rotation angle θ on the frictional strength of the clutch device 10. However, it can be important to acknowledge that the analysis is based on the small angle approximation described above, while the tested clutch device 10 in the experiments has a rotation angle θ of about 0.6. Further, the experimental clutch device 10 differs from the analysis in terms of the potential looser packing of the sheets 18, 38, 58, 78 at the sewing points (fold lines 24, 44, 64, 84) under a small load.

Finally, to estimate the energy consumption of the clutch device 10 during the switching process, the energy difference of the clutch device 10 between the locked and unlocked states can be evaluated. At least because the switching can be achieved, for example, by rotating the pluralities of sheets 16, 36, 56, 76, the energy difference between the two states can be considered as the sum of the bending energy differences for all sheets 18, 38, 58, 78. By adopting the same continuous approximation used previously, the total bending energy difference can be calculated using Equation 12:

$$Δ U = 2 M {\int }_{0}^{1} Δ {u}_{z} d z = 2 M ( 3 D \frac{( 2 δ - θ {L}_{f} ) ε M}{{L}_{f}^{3}} + 2 D \frac{3 {δ}^{2} - 3 δ θ {L}_{f} + {θ}^{2} {L}_{f}^{2}}{{L}_{f}^{3}} ) , \qquad ( 12 )$$

and is depicted in FIG. 8H. The plot shows that the bending energy difference between the locked and unlocked states is on the order of a few mJ.

The method of unlocking the clutch device 10 through bending of the layers provides an opportunity to leverage a bistable or multistable mechanism for clutch control. As a demonstration of this concept, the control of the state of the clutch device 10 was demonstrated by manipulating the positions of rubber bands (i.e., clamps 94, 97), as depicted in FIGS. 4A and 4B. When the rubber bands 94, 97 are placed near the sewing points (fold lines 24, 44, 64, 84), the clutch device 10 remains in the locked state. In this configuration, these rubber bands 94, 97 apply a small compressive force to the sheets 18, 38, 58, 78 near the fold lines 24, 44, 64, 84, but the magnitude of this force is negligible compared to the compressive force applied by the twine 32, 52, 72, 92 to the sheets 18, 38, 58, 78 during loading. Conversely, unlocking the clutch can be accomplished by rolling the rubber bands 94, 97 towards the second terminal ends 23, 43, 63, 83, effectively clamping the distal end portions 22, 42, 62, 82 and generating a rotation to the non-overlapping region 20, 40, 60, 80 of the sheets 18, 38, 58, 78. The rubber bands 94, 97 retain their positions, maintaining the clutch device 10 in the desired state without continuous energy consumption.

In certain robotic devices in which the clutch device 10 is utilized, a dedicated resetting mechanism that keeps the stacks 14, 55 from separating too far may not be essential for the clutch device 10. The sliding of the stacks 14, 55, in such instances, naturally follows the motion of the part to which it is attached. For example, in electroadhesive clutch-based haptics, the clutch device 10 can be anchored between the fingertip and wrist, sliding in response to the flexion of the finger and engaging into a locked state at the current position upon demand.

However, in some other cases, the implementation of a resetting mechanism can be advantageous to achieve a consistent and reliable clutch behavior during cyclic operation. Typically, this resetting mechanism can involve integrating a spring element and/or a resetting cord member 99 in parallel or substantially parallel with the central longitudinal axis 11 of the clutch device 10. The elasticity of the resetting cord member 99 can return the stacks 14, 55 to their starting, interleaved, locked arrangement after being pulled apart in an unlocking process. In other words, after the stacks 14, 55 have been unlocked and a load is applied to slide the stacks 14, 55 away from each other, the resetting cord member 99 pulls the stacks 14, 55 back together such that the clutch device 10 returns to its original, undeformed geometry. This return to the original geometry can occur after the load applied to pull apart the stacks 14, 55 is either reduced or removed.

In one non-limiting example, an elongated rubber band can serve as the resetting cord member 99, as depicted in FIGS. 13C and 15E. In some embodiments, the resetting cord member 99 can extend between and be coupled to the clamps 94, 97, as shown in FIG. 15E, while in other embodiments, the resetting cord member 99 can be coupled to any other portion of the stacks 14, 55 that moves relative to the other stack 14, 55. For example, the resetting cord member 99 may include a first end 99A coupled to the first plurality of sheets 16 and a second end 99B coupled to the second plurality of sheets 36. In some embodiments, the resetting cord member 99 prevents the stacks 14, 55 from moving beyond a predetermined stack separation distance to, for example, prevent the stacks 14, 55 from being completely separated and disengaged (i.e., retaining some interleaving). In some embodiments, the resetting cord member 99 can be rigid so as to prevent the complete separation, but will not return the stacks 14, 55 to the original starting arrangement due, at least in part, to lacking elasticity.

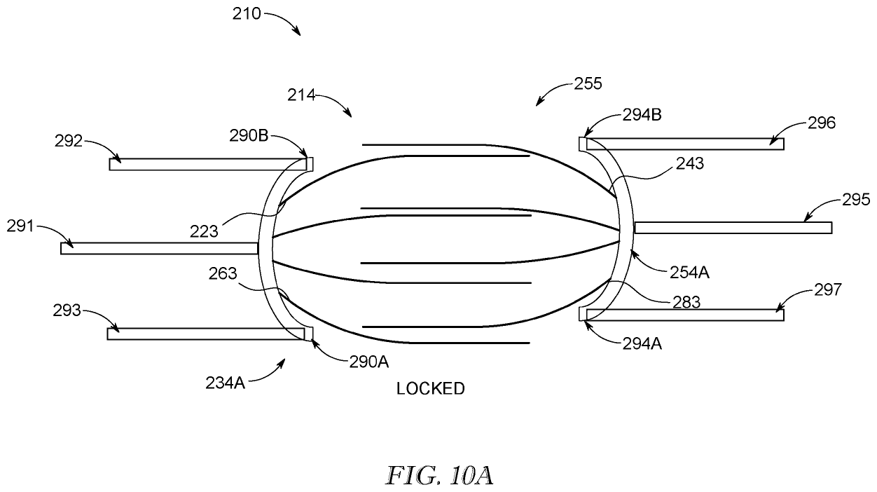

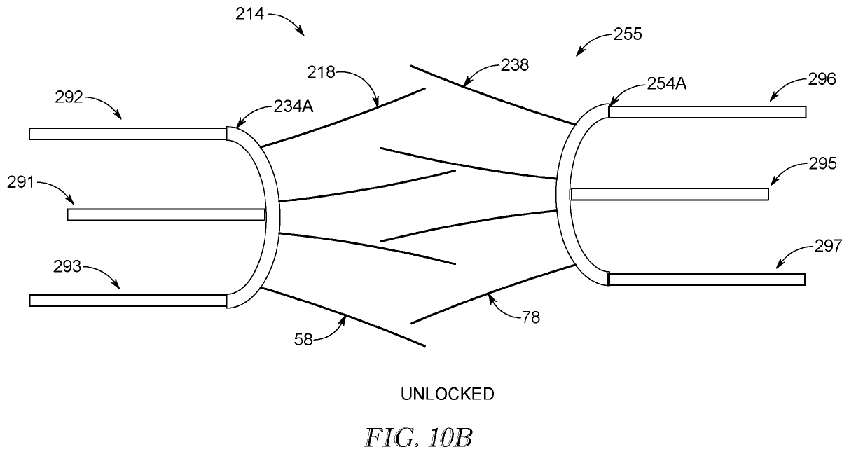

FIGS. 15F and 15G schematically illustrate an embodiment of the clutch device 10″ that does not employ a resetting cord member 99, and what may occur, for example, in the event the stacks 14, 55 separate to great a distance. The clutch device 10″ is formed similarly to the clutch devices 10, 10′ described herein and thus the same reference numerals are utilized but for the additional prime symbol (′) to represent like components. It is noted that reference numerals 24″, 44″, 64″, 84″ may refer to the fold lines 24, 44, 64, 84 described above, and reference numerals 34A′ and 54A′ may refer to the bending actuation members 234A, 254A described with reference to FIGS. 10A-12C below. In such an embodiment, when the stacks 14, 55 are brought back together after having been completely separated, as shown in FIG. 15F, the reengagement may not be perfect, with some sheets 18″, 38″, 58″, 78″ having multiple opposing sheets 18″, 38″, 58″, 78″ disposed therebetween, as shown in FIG. 15G. This may reduce locking strength in some scenarios. It is noted that such arrangements may still be considered to include “interleaved” sheets, as the term is used herein.

It is noteworthy that one of the primary motivations for employing a clutch in a robotic system is to implement clutched elastic actuators, where clutches work in conjunction with spring components. Such a spring, when used in parallel with the clutch, inherently serves the dual purpose of providing the restoring force for the clutch without necessitating further modification.

As evidenced by the description herein, the clutch devices of the present disclosure provides numerous advantages over prior clutch devices. As described above, an optimized clutch should possess several key characteristics, including having a high locking force, low energy consumption, compactness, cost-effectiveness, robustness, rapid switching time, and being lightweight. Moreover, a clutch may require being able to unlock while under load, and be lockable in any position. The clutch devices disclosed herein can unlock when subjected to tension. Moreover, the disclosed clutch devices do not require continuous energy consumption to maintain a specific state when a bistable switching mechanism is implemented. Additionally, the energy consumed for switching is very small, primarily associated with supplying the bending energy of the layers. Furthermore, the disclosed clutch devices can be locked in any position along the range of allowable extension given the overlapping between two stacks, providing flexibility in its engagement position. Even further, the disclosed clutch devices are compact, lightweight (paper or similar materials can be used for the sheets), and provide high locking force. For example, the two stacks of interleaved layers of the present disclosure can achieve a locking force of about 550 N with about 100 mm length, about 25 mm width, and about 12 mm thickness (the thickness can equal the layer thickness multiplied by the layer number). The disclosed clutch devices are also cost-effective and inexpensive to manufacture, operate, and maintain.

FIG. 9 shows a quantitative comparison of the weight and energy consumption performance of the disclosed clutch devices with prior clutches based on different mechanisms. An “x” denotes a linear clutch, while an “ ” signifies a rotary clutch, with force calculated by dividing torque by the radius of the clutch plate. As can be seen in FIG. 9, only the disclosed clutch devices and the mechanical latches are shown as dashed lines because they can maintain a prescribed state without continuous power consumption. Therefore, the disclosed clutch devices have significantly lower energy consumption than electroadhesive, electromagnetic, and magnetorheological clutches. An exemplary 60-layer paper-based clutch device weighs 9 g and possesses a locking force of around 550 N, resulting in a force/mass ratio approximately one order of magnitude higher than the reported performance of the mechanical latch. Furthermore, it surpasses the force/mass ratio of all prior clutches shown in FIG. 9. It is important to note that the locking force of the disclosed clutch devices scales exponentially with the square of the number of layers, as described by Equation 4, while the weight scales linearly with the number of layers. Therefore, it is feasible for the disclosed clutch devices to achieve an even higher force/mass ratio. Accordingly, the disclosed clutch devices hold promise for use in robotics, exoskeletons, prosthetics, and other mechanical systems that require switchable engagement, at least some of which are described herein. Further, a person skilled in the art, in view of the present disclosures, will appreciate alternate materials and/or fabrication processes (e.g., 3D printing) that can be used in conjunction with the present disclosures.

Another embodiment of a clutch device 210 is shown in FIGS. 10A-12C. The clutch device 210 is similar to the clutch devices 10, 10′, 10″ described herein. Accordingly, similar reference numbers in the 200 series indicate features that are common between the clutch device 210 and the clutch devices 10, 10′, 10″. The descriptions of the clutch devices 10, 10′, 10″ are incorporated by reference to apply to the clutch device 210, except in instances when they conflict with the specific description and the drawings of the clutch device 210.

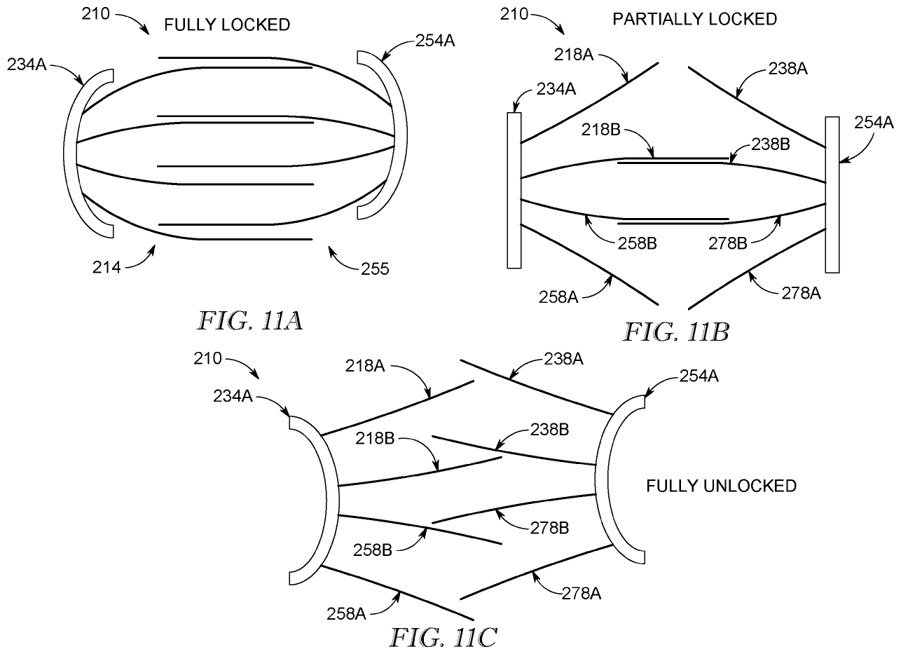

As can be seen schematically in FIGS. 10A-11C, the clutch device 210 can be formed substantially similarly to the clutch devices 10, 10′, 10″ described above. The clutch device 210 differs at least in that, instead of including bent distal end portions 22, 42, 62, 82, the second terminal ends 223, 243, 263, 283 are coupled directly to a bendable actuation member 234A, 254A. The bendable actuation member 234A, 254A may include an elongated platform that the second terminal ends 223, 243, 263, 283 are directly coupled to and that is bendable or in some way deformable. In some embodiments, the bendable actuation member 234A, 254A is configured to be actuated so as to bend from the locked position shown in FIG. 10A, in which the clutch device 210 is in a locked state, to the unlocked position shown in FIG. 10B, in which the clutch device 210 is in an unlocked state, and any position therebetween.

As shown in FIGS. 10A and 10B, the clutch device 210 may further include external actuators 290, 294, which can each include a central fixture bar 291, 295 and linear actuators 292, 293, 296, 297 operably coupled to opposing ends 290A, 290B, 294A, 294B of the bendable actuation members 234A, 254A. The linear actuators 292, 293, 296, 297 may be actuated by any known means in the art, such as electronically, pneumatically, hydraulically, and the like. As shown in FIG. 10B, while the central fixture bar 291, 295 keeps the center of the respective bendable actuation members 234A, 254A in place, the linear actuators 292, 293, 296, 297 can be actuated so as to move the opposing ends 290A, 290B, 294A, 294B of the bendable actuation members 234A, 254A away from the stacks 214, 255. This causes bending of the bendable actuation members 234A, 254A away from the stacks 214, 255.

The bending of the bendable actuation member 234A, 254A away from the stacks 214, 255 can cause the same rotation in the pluralities of sheets 216, 236, 256, 276 of the stacks 214, 255 as the rotation caused by the clamping of the bent distal end portions 22, 42, 62, 82 of the clutch device 10. The bendable actuation member 234A, 254A can provide an advantage in that the actuation member 234A, 254A can be arranged in any position between those shown in FIGS. 10A and 10B. For example, FIG. 11A shows a fully locked position and FIG. 11C shows a fully unlocked position of the bendable actuation member 234A, 254A. FIG. 11B shows a middle position of the bendable actuation member 234A, 254A in which the clutch device 210 is partially locked, in particular because the bendable actuation member 234A, 254A has been bent enough to allow the outer sheets 218A, 238A, 258A, 278A to disengage while maintaining engagement of the middle sheets 218B, 238B, 258B, 278B.

This control of the bendable actuation member 234A, 254A allows for, by way of example, precise control of the stiffness of the clutch and/or the maximum locking force the clutch device 210 can sustain. In other words, because the extent of the bending of the actuation members 234A, 254A is selectable, the number of sheets 218, 238, 258, 278 that are interleaved can be selectable or controllable at any given juncture of rotation. In some embodiments, the bendable actuation member 234A, 254A can be rotated to adjust the stiffness of the clutch and/or the maximum locking force the clutch device 210 can sustain. A threshold number of sheets 218, 238, 258, 278 can be predetermined to set a limit for how many sheets can be disengaged before the clutch device 210 becomes unlocked. This can be based on many factors such as those described above, including number of sheets, the force pulling the two stacks 14, 55 apart, and the like. A person skilled in the art will understand that the threshold number of sheets can be based on the amount of locking force required of the particular application, and in some embodiments, the threshold number of sheets can bear a mathematical functional relationship with the amount of locking force required of the particular application. For example, locking force reduces continuously as the number of locked layers reduces. In embodiments in which the layers can be precisely controlled layer-by-layer, such as with the bendable actuation members 234A, 254A, the stiffness and the maximum locking force can change in a layer-by-layer manner.

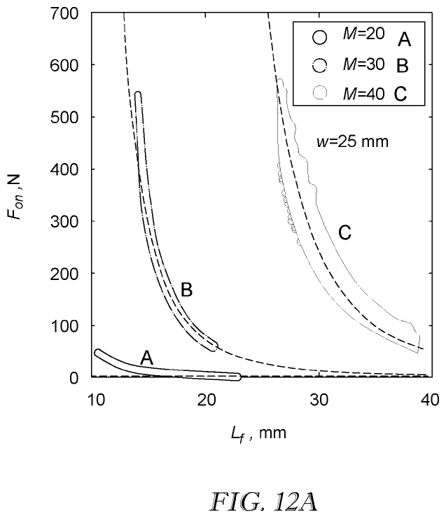

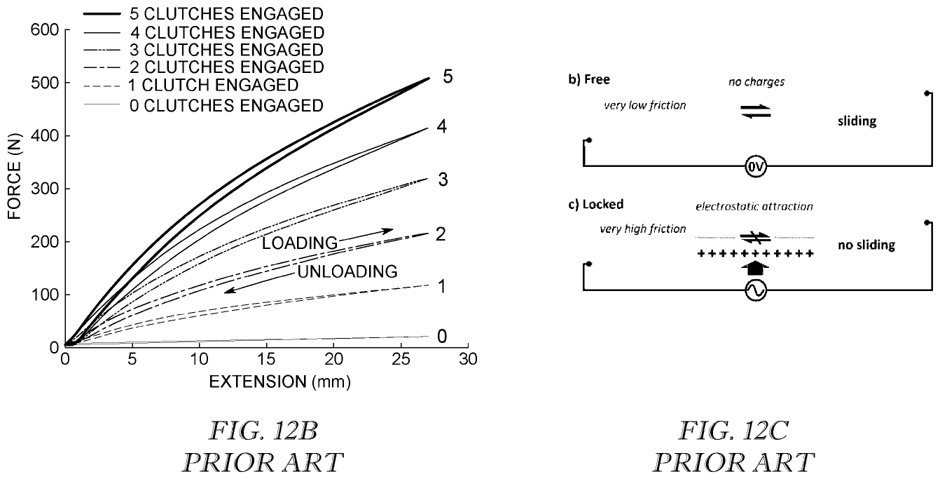

FIG. 12A shows that the locking strength (Fₒₙ) and the tensile stiffness depend, at least in part, on the number of layers engaged (M). This data can be applied to the clutch device 210 described above. By tuning M, Fₒₙ, which can be tuned on demand or be predetermined, rehabilitation to control resistance can be improved. When M layers are engaged, the tensile stiffness of the clutch device 210 can be equivalent of a block of M layers in parallel. As such, the number of layers M can be tuned to adjust the stiffness of the clutch device 210.

As a comparison to the clutch device 210, FIG. 12B shows the locking strength versus separation distance of an exemplary prior single layer (i.e. one layer or sheet on a side as FIG. 12C shows) clutch device, and FIG. 12C shows an example of a prior clutch device that is not based on self-locking, but instead is operated via electrostatic attraction between two layers, which then generate friction based on normal electrostatic attraction. Similar multiple layer clutches also use this technique, in particular multiple iterations of this “single layer clutch” simply put in parallel, there being no interaction between each “single layer clutch.” The total force and tensile stiffness are simply added together, which are the results shown in FIG. 12B.

Another embodiment of a clutch device 310, 310′ is shown in FIGS. 13A-14. The clutch device 310 is similar to the clutch devices 10, 10′, 10″, 210 described herein. Accordingly, similar reference numbers in the 300 and 300′ series indicate features that are common between the clutch device 310, 310′ and the clutch devices 10, 10′, 10″, 210. The descriptions of the clutch devices 10, 10′, 10″, 210 are incorporated by reference to apply to the clutch device 310, 310′, except in instances when they conflict with the specific description and the drawings of the clutch device 310, 310′.

As can be seen schematically in FIG. 13A, the clutch device 310 can be formed substantially similarly to the clutch devices 10, 210 described above. The clutch device 310 differs at least in that, instead of including two pluralities of sheets 16, 56, 36, 76 pulled together in each stack 14, 55, only a “half-stack” arrangement is used in which only single pluralities of sheets 316, 336 make up the stacks 314, 355. Moreover, the clutch device 310 further differs from the clutch devices 10, 210 at least in that the stacks 314, 355 rest on a platform 328, which can be a single platform 328, as shown in FIGS. 13A and 13B, or multiple platforms, such as dual platforms 328A and 328B that are configured to move relative to each other when the clutch device 310 is locked and unlocked, as shown in FIGS. 13C and 13D.

As described above, in this “half-stack” arrangement, the pluralities of sheets 316, 336 apply the normal force to the platform 328, while the reactionary normal force from the platform 328 is applied to the sheets 316, 336. This balance creates the friction force required to keep the stacks 314, 355 of sheets 316, 336 locked. In some embodiments, as shown in FIG. 13A, the lowermost sheets 318, 338 can rest on the platform 328 and all of the sheets 318, 338 can be held in tension. This is similar to how the sheets 18, 38 described above are held in tension, in particular, for example, via a plurality of cords 332, 352, thus creating the normal and friction forces responsible for holding the clutch device 310 in the locked arrangement. In some embodiments, the plurality of cords 332, 352 that hold the sheets 318, 338 in tension may be coupled to an external fixture (not shown).

In order to unlock the stacks 314, 355, the same rotation can be applied to the pluralities of sheets 316, 336 of the stacks 314, 355 as the rotation caused by the clamping of the bent distal end portions 22, 42, 62, 82 of the clutch device 10. Specifically, the sheets 318, 338 can include bent distal end portions 322, 342 formed the same as the distal end portions 22, 42 shown in FIGS. 3A and 4A, and schematically as shown in FIGS. 6A and 6B. Similar to the rotational movement shown in FIG. 6B, the bent distal end portions 322, 342 of the sheets 318, 338 can be moved in the downward direction 396, 398 so as to rotate the outer portions of the sheets 318, 338 about their respective fold lines 324, 344, and then the stacks 314, 355 can be unlocked and separated, as shown in FIG. 13B.

In some embodiments, as shown in FIGS. 13C and 13D, the cords 332′, 352′ can be coupled directly to the platform 328, which, in such embodiments, can be split into first and second platforms 328A′, 328B′. This coupling creates the tension in the stacks 314′, 355′ so as to create the normal and frictional forces required to lock the clutch device 310′. The bent distal end portions of the sheets 322′, 342′ can be moved so as to rotate the outer portions of the sheets 318′, 338′ and thus unlock the device 310′. In doing so, the two platforms 328A′, 328B′ can be moved away from each other, as each platform 328A′, 328B′ includes a respective one of the stacks 314′, 355′ coupled thereto. In some embodiments, the platforms 328A′, 328B′ may be connected via a resetting cord member 399′, similar to the resetting cord member 99 discussed above. The resetting cord member 399′ may be a resilient, spring-like member that prevents the platforms 328A′, 328B′ from extending beyond a point at which the sheets 318, 338 would no longer overlap, and may also return the platforms 328A′, 328B′ back to the starting position where the clutch device 310′ can be relocked.

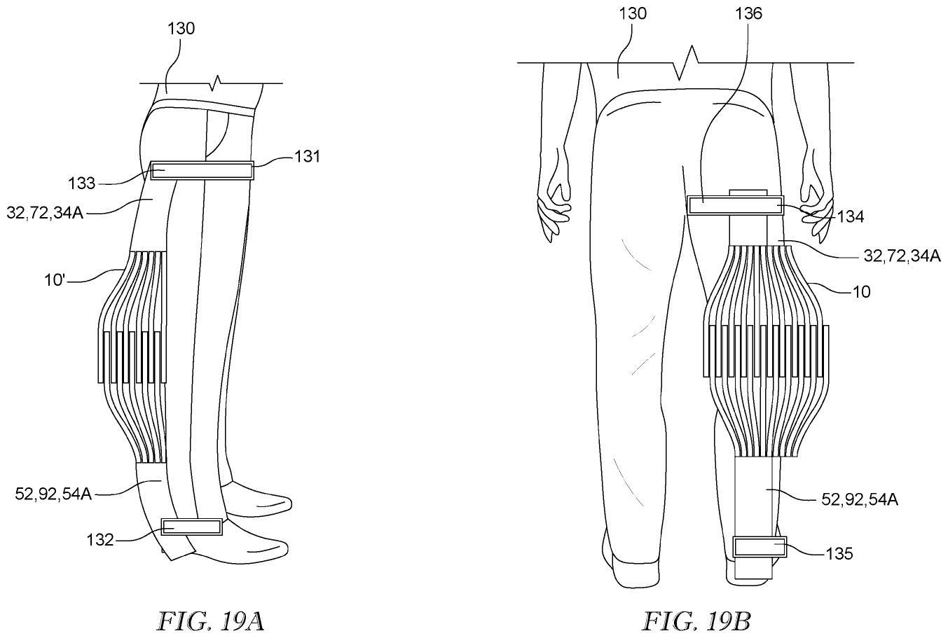

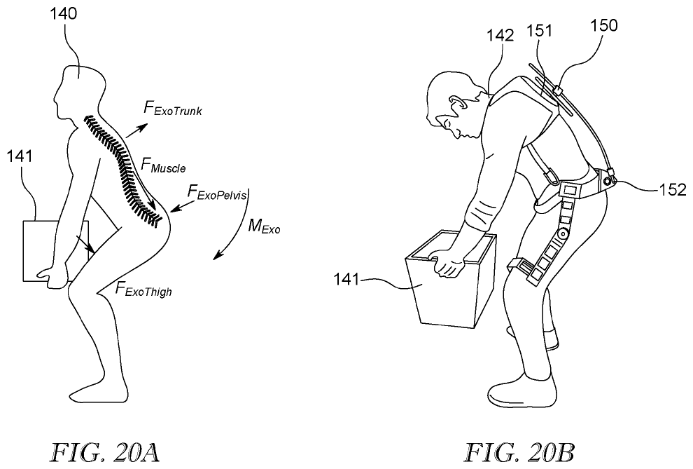

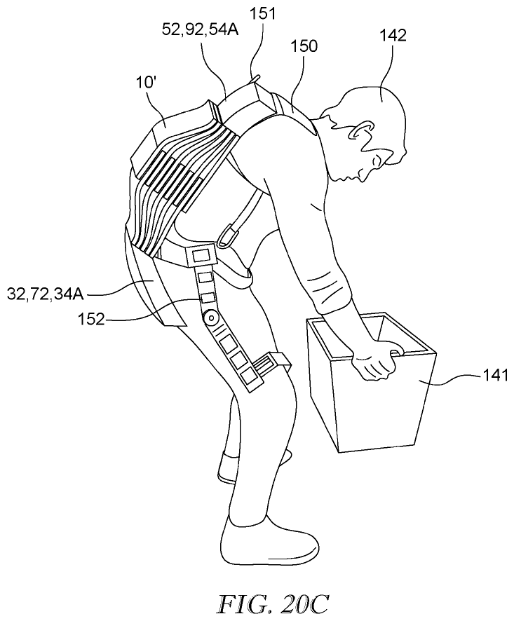

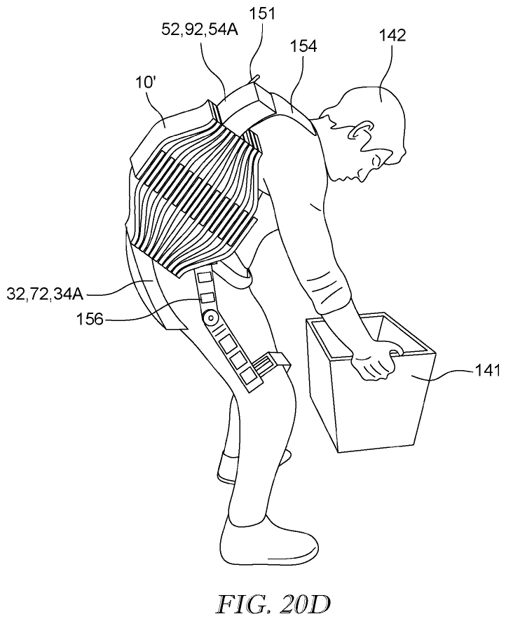

This “half-stack” arrangement of the clutch device 310 enables the device 310 to be bent about a top surface of a curved substrate or platform (i.e. in scenarios in which the platform 328 may be curved and the cords 332, 352 are pulled downwardly in the downward directions 396, 398) while still holding the locked position. In some embodiments, the curved substrate or platform on which the stacks 314, 355 are resting on may serve as the platform 328. The pluralities of sheets 316, 336 of the stacks 314, 355 typically will not be disengaged, even when on a curved platform, until the distal end portions 322, 342 are moved and the sheets 318, 338 are rotated, as described above. This may be particularly useful in applications that require the clutch device 310 to curve about a surface, as shown in, for example, FIG. 20C.

### Applications of Clutch Devices

Clutch devices of the present disclosures can be implemented in a variety of contexts where a person skilled in the art, in view of the present disclosures, will appreciate such clutch devices would be useful. This can include instances where clutch devices of the present disclosures can be swapped into known implementations of existing clutch devices, as well as new applications of clutch devices in view of the designs and functionality of the disclosed clutch devices. This can include, but is not limited, to use in medical devices, textiles, robotics, haptics, and other uses provided for herein or otherwise known to those skilled in the art.

One non-limiting potential application of the clutch device 10 disclosed herein is shown in FIGS. 15A-15E as part of a medical device. To showcase this application of the clutch device 10, a prototype of a switchable back brace posture corrector 100 featuring a motorized switchable clutch was developed, as shown in FIG. 15A-15E. As shown in FIGS. 15A and 15B, the switchable posture corrector 100 was designed as a wearable device, with the addition of a cover 14C and a segment of twine (i.e. resetting cord member 99) to prevent the stacks 14, 55 from separating axially. The tubing cover 14C served to enclose and protect the clutch device 10. The additional twine 99 was connected between a motorized robotic claw 102 and an adjustable tie 103 to a lower stack of paper to prevent the clutch from extending beyond 105 mm. Locking and unlocking of the clutch was controlled by the robotic claw 102 mounted at its top end as illustrated by FIG. 15B. While being worn on the individual's back, the clutch device 10 offered resistance to motion while in the locked state. Conversely, when in the unlocked state, the clutch device 10 permitted free extension.

To fabricate the switchable posture corrector 100, a 60-layer clutch device 10 was used to replace the center spine of a commercially available back brace posture corrector (e.g., Selbite Posture Corrector, Amazon). The clutch device 10 was affixed using clips (e.g., Small binder clips, Amazon) and adjustable ties (e.g., Nylon cable tie, McMaster-Carr) as shown in FIG. 15B. To enable the unlocking of the clutch device 10, the robotic claw 102 is actuated by a micro servo motor (e.g., Micro gripper kit, Pololu Robotics and Electronics) was utilized. The motorized claw was mounted onto a 3D-printed fixture (e.g., Ultimaker 2+, Ultimaker) using screws. The fixture was then sewn to the posture corrector at a location where the closing of the claw would clamp the distal ends of the clutch together, as depicted in FIG. 4A. The opening and closing of the motorized claw were controlled by a microcontroller (e.g., Arduino Uno, Arduino). For this demonstration, only one robotic claw was implemented to actuate one side of the clutch device 10.

To enclose and protect the clutch device 10, a tubing cover 14C (e.g., 50 mm inner diameter and 175 mm long) made of paper was added. Moreover, to restrict the sliding distance of the clutch and prevent complete disengagement during use, an approximately 150 mm long twine 99 was fastened between the claw 102, controlling the upper stack of paper, and the adjustable tie 103 securing the lower stack of paper.

As described above, a resetting mechanism can involve integrating a spring element and/or a resetting cord member 99 in parallel or substantially parallel with the central longitudinal axis 11 of the clutch device 10, as shown in FIG. 15E. FIGS. 15F and 15G depict an embodiment in which such a resetting cord member 99 is not utilized.

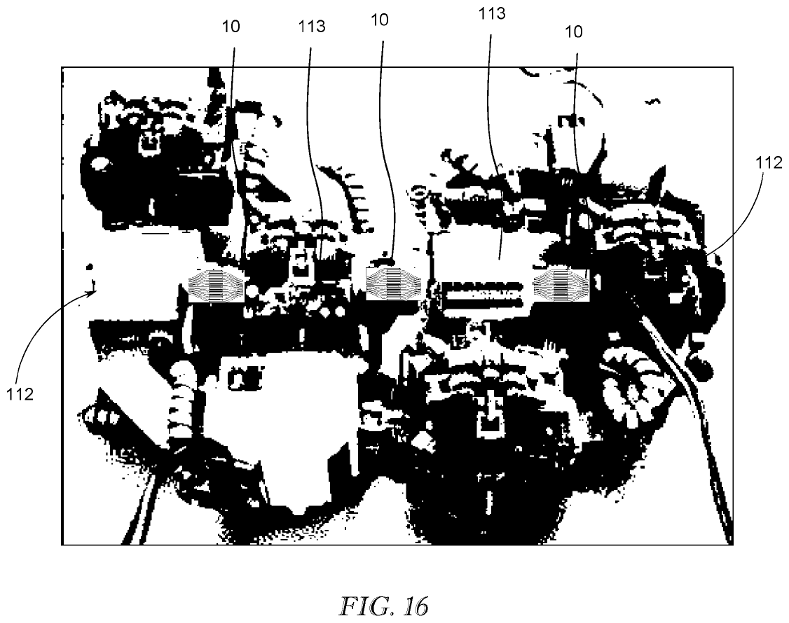

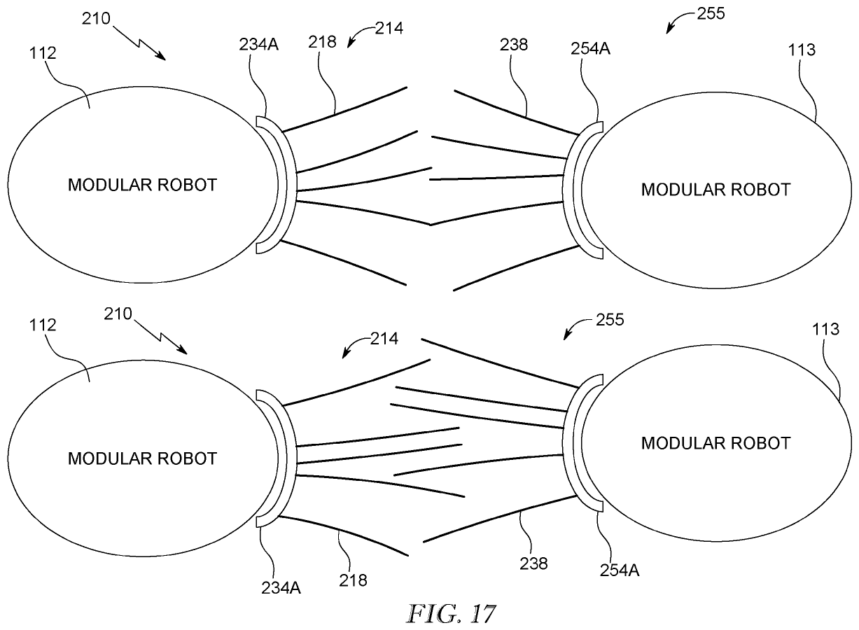

FIGS. 16 and 17 show views of an application of the clutch device 10 of FIGS. 9E and 9F, in particular a clutch device 10 that does not include a resetting cord member. Such a clutch device 10 can be utilized as a docking mechanism to connect and disconnect modular robots, such as between the robots 112, 113 shown in FIG. 16. The robots 112, 113 can use a series of latching, rotation, and/or unlatching to self-reconfigure, the latching being accomplished via the clutches described herein. Each module can have four degrees of rotational freedom and can mate with its neighbors using, for example, gendered connectors.

As shown in FIG. 17, the application of the modular robots 112, 113 of FIG. 16 can additionally or alternatively utilize the clutches 210 shown in FIGS. 10A-11C, in particular the clutches having bendable actuation members 234A, 254A. The clutches of this application may also not include a resetting cord member, such as the clutch 10″ shown in FIGS. 15F and 15G, so as to allow the stacks to be completely separated, thus allowing the modular robots 112, 113 to be completely separated from each other. FIG. 17 also shows an example of when more than a single sheet is interleaved between adjacent sheets while still being configured to maintain locking strength when locked.

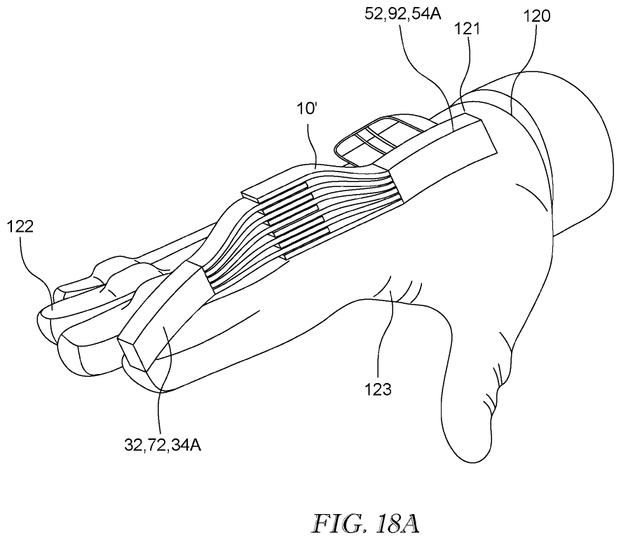

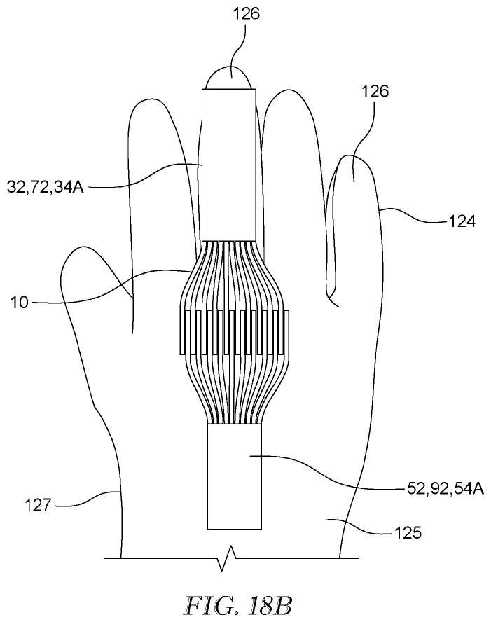

FIG. 18A shows a haptic device 120 having a clutch device of the present disclosure, in particular the “half stack” clutch device 10′ of FIGS. 6A and 6B incorporated into the haptic glove 120. A person skilled in the art will appreciate that while the illustrated embodiment uses the clutch device 10′, other clutch devices of the present disclosure, or derivable in view of the present disclosure, can be used in lieu of, or in addition to, the clutch device 10′. The glove 120 has a typical glove configuration, which includes a base portion 121, a plurality of fingers 122 extending from the base portion 121, and a palm portion 123 opposite of the base portion 121. As shown, the clutch device 10′ is coupled to an end of at least one finger 122 and the base portion 121. In particular, as described above, in some embodiments, the ends of the cords 32, 52, 72, 92 or the tensioning members 34A, 54A may be attached to the finger 122 and the base portion 121. The clutch device 10′ may be coupled to any single finger, or in some embodiments, multiple clutch devices 10′ can be coupled to multiple fingers 122. The clutch device 10′ shown in FIG. 18A utilizes a “half-stack” configuration between an end of a finger 122 of the base 121 of the glove 120.

An alternative embodiment of a haptic glove 124 is akin to the haptic glove 120 of FIG. 18A, and thus includes a base portion 125, a plurality of fingers 126 extending from the base portion 125, a palm portion 127 opposite the base portion 125, and a clutch device 10 incorporated into the glove 124. Unlike the glove 120, the illustrated clutch device 10 utilizes a “full-stack” configuration between an end of a finger 126 of and the base 125 of the glove 124. In some embodiments, the ends of the cords 32, 52, 72, 92 or the tensioning members 34A, 54A may be attached to the finger 126 and the base portion 125.

FIG. 19A shows an exoskeleton 131 attached to a human user 130 having a clutch device of the present disclosure, in particular the “half stack” clutch device 10′ of FIGS. 6A and 6B incorporated into the exoskeleton 131. A person skilled in the art will appreciate that while the illustrated embodiment uses the clutch device 10′, other clutch devices of the present disclosure, or derivable in view of the present disclosure, can be used in lieu of, or in addition to, the clutch device 10′. The exoskeleton 131 may include a lower attachment member 132 coupled to a heel of the foot of the user 130, and an upper attachment member 133 coupled to an upper leg portion of the user 130. As shown and as described above, in some embodiments, the ends of the cords 32, 52, 72, 92 or the tensioning members 34A, 54A of the clutch device 10′ may couple the lower attachment member 132 to the upper attachment member 133. The clutch device 10′ may be utilized on one or both legs of the user 130. The clutch device 10′ is configured to selectively lock and unlock so as to provide high strength support to the lower body of the user 130.

An alternative embodiment of an exoskeleton 134 is akin to the exoskeleton 131 of FIG. 19A, and thus includes a lower attachment member 135 coupled to a heel of the foot of the user 130, and an upper attachment member 136 coupled to an upper leg portion of the user 130, and a clutch device 10 incorporated into the exoskeleton 134. Unlike the exoskeleton 131, the illustrated clutch device 10 utilizes a “full-stack” configuration between the lower and upper attachment members 135, 136. In some embodiments, the ends of the cords 32, 52, 72, 92 or the tensioning members 34A, 54A may be attached to the lower and upper attachment members 135, 136.

FIG. 20A is a schematic view of a human user lifting an object and the forces acting on the body during this motion, which include back muscles contracting (F_{Muscle}). This force acts over a small lever arm (r_{Lever}) about parallel to the spine, which causes the spine to compress. An exoskeleton can help reduce these forces, by applying forces on the trunk (F_{ExoTrunk}), pelvis (F_{ExoPelvis}), and thigh (F_{ExoThigh}), thus producing an extension moment (M_{Exo}).

FIG. 20B is a side view of an exoskeleton 150 configured to aid the human user in lifting the object and that can utilize a clutch device as described herein. In some embodiments, the exoskeleton 150 may include an upper harness 151 configured to attach to the upper torso of a user 142 and a lower harness 152 configured to attached to a pelvic area of the user 142.

FIG. 20C shows the exoskeleton 150 attached to the human user 142 having a clutch device of the present disclosure, in particular the “half stack” clutch device 10′ of FIGS. 6A and 6B incorporated into the exoskeleton 150. A person skilled in the art will appreciate that while the illustrated embodiment uses the clutch device 10′, other clutch devices of the present disclosure, or derivable in view of the present disclosure, can be used in lieu of, or in addition to, the clutch device 10′. As shown and as described above, in some embodiments, the ends of the cords 32, 52, 72, 92 or the tensioning members 34A, 54A of the clutch device 10′ may couple the upper harness 151 to the lower harness 152. The clutch device 10′ is configured to selectively lock and unlock so as to provide high strength support to the user's 142 back when lifting objects 141. Moreover, the amount of force applied by the clutch device 10′ can be adjusted based on how many layers are engaged, thus allowing for precise control of how much force is applied to the exoskeleton 150. Even further, as described above, the flexibility required by the back of a user 142 lifting an object can be accommodated by the “half-stack” configuration of the clutch devices described herein, and the rounding of the user's 142 back can even unlock the clutch 10′ as shown in embodiments such as those shown in FIGS. 13-14B.

An alternative embodiment of an exoskeleton 154 is akin to the exoskeleton 150 of FIG. 20A, and thus includes an upper harness 155, a lower harness 156, and a clutch device 10 incorporated into the exoskeleton 154. Unlike the exoskeleton 150, the illustrated clutch device 10 utilizes a “full-stack” configuration between the upper and lower harnesses 155, 156. In some embodiments, the ends of the cords 32, 52, 72, 92 or the tensioning members 34A, 54A may be attached to the upper and lower harnesses 155, 156.

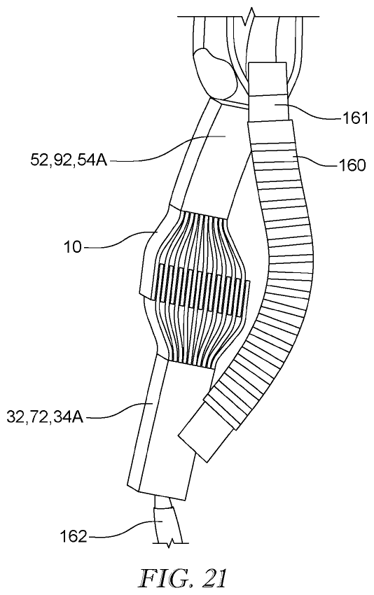

FIG. 21 shows how the clutch devices of the present disclosure can be integrated into a “soft finger” device 160, in particular by having the clutch device 10 integrated into it, the clutch device 10 being in a “full-stack” configuration. In particular, the clutch device 10 can be coupled to terminal ends 161, 162 of the soft finger device 160 via ends of the cords 32, 52, 72, 92 or the tensioning members 34A, 54A.

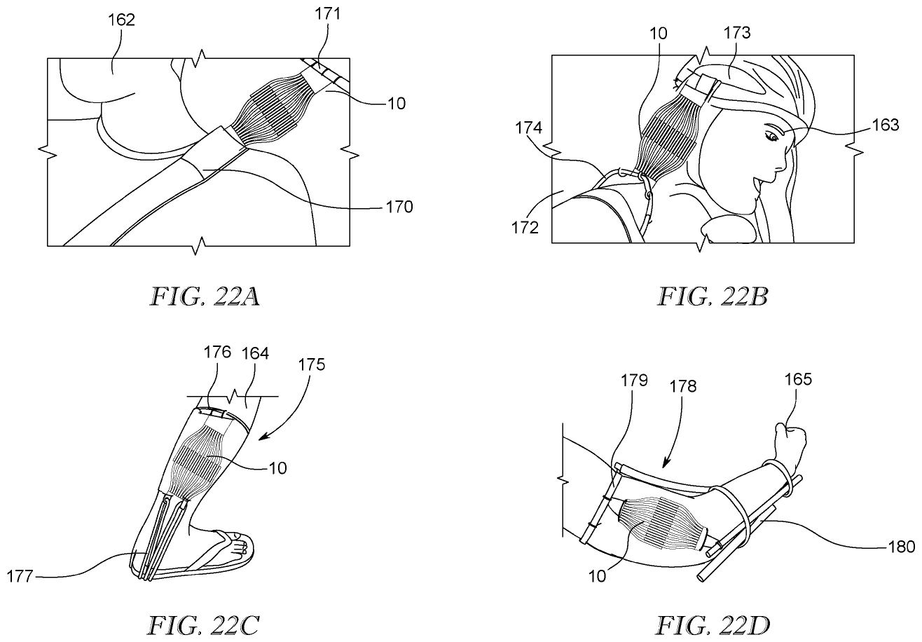

FIGS. 22A, 22B, 22C, and 22D show how the disclosed clutch devices can be integrated into various human applications. It is noted that any of the embodiments shown in FIGS. 22A-22D may utilize the cords 32, 52, 72, 92 or the tensioning members 34A, 54A to couple the clutch devices 10, 10′ to the respective user component.

As one example, as shown in FIG. 22A, a damping seat belt 170 can utilize a clutch device according to the present disclosure. The seat belt 170 can be coupled to a base member 171 via one of the clutch devices described herein, such as, for example, the “half-stack” clutch device 10′ or the “full-stack” clutch device 10. Damping of the movement of the user 162 during impact can be improved via selective engagement of the clutch device 10, 10′.

As a further non-limiting example, a damping neck guard 172 can utilize a clutch device according to the present disclosure. The damping neck guard 172 can be coupled to a helmet 173 and a base member 174 on the user's 163 upper torso via one of the clutch devices described herein, such as, for example, the “half-stack” clutch device 10′ or the “full-stack” clutch device 10. Damping of head movement of the user 163 during impact can be improved via selective engagement of the clutch device 10, 10′.

As another non-limiting example, an ankle exoskeleton 175 configured as a clutch can utilize a clutch device according to the present disclosure. The ankle exoskeleton 175 can be coupled to a user's 164 leg around the calf area via an upper attachment member 176 and near the heel of the user's 164 foot via a lower attachment member 177. The upper and lower attachment members 176, 177 may be coupled to each other via one of the clutch devices described herein, such as, for example, the “half-stack” clutch device 10′ or the “full-stack” clutch device 10. Selective engagement of the clutch device 10, 10′ can provide additional support to the user's 164 ankle area.

As yet another non-limiting example, a flexion muscle rehabilitation device 178 configured as a brake 175 can utilize a clutch device according to the present disclosure. The flexion muscle rehabilitation device 178 can be coupled to a user's 165 upper arm via an upper attachment member 179 and near the user's 165 forearm via a lower attachment member 180. The upper and lower attachment members 179, 180 may be coupled to each other via one of the clutch devices described herein, such as, for example, the “half-stack” clutch device 10′ or the “full-stack” clutch device 10. Selective engagement of the clutch device 10, 10′ can provide additional support to the flexion muscle rehabilitation device 178.

Examples of the above-described embodiments can include the following:
1. A clutch device, comprising:
   a first plurality of flexible members;
   a second plurality of flexible members interleaved with the first plurality of flexible members;
   at least one overlapping region defined in an area in which flexible members of each of the first and second pluralities of flexible members overlap each other; and
   a plurality of non-overlapping regions defined in areas on opposing sides of the overlapping region, the plurality of non-overlapping regions being regions in which the flexible members of each of the first and second pluralities of flexible members do not overlap each other,
   wherein, in a locked arrangement of the clutch device, an outer portion of each of the plurality of non-overlapping regions extends at a first angle relative to a plane defined by a corresponding overlapping region of the at least one overlapping region such that the first and second pluralities of flexible members engage and interlock with each other and prevent the first and second pluralities of flexible members from sliding with respect to each other, and
   wherein the outer portions of each of the plurality of non-overlapping regions are configured to be moved to a second angle relative to the plane defined by the at least one overlapping region to bend the flexible member outwardly, the second angle being different than the first angle, and such movement causing the clutch device to move into an unlocked arrangement in which the first and second pluralities of flexible members are disengaged from each other so as to allow the first and second pluralities of flexible members to slide with respect to each other.
2. The clutch device of example 1, wherein the second angle is greater than the first angle.
3. The clutch device of examples 1 or 2,
   wherein each flexible member of the first and second pluralities of flexible members includes a first terminal end that overlaps an opposing flexible member of the other of the first and second pluralities of flexible members,
   wherein each flexible member of the first and second pluralities of flexible members further includes a second terminal end opposite the first terminal end, and
   wherein a pivot axis is defined on each flexible member of the first and second pluralities of flexible members about which the respective plurality of flexible members is configured to be rotated.
4 The clutch device of example 3, wherein each flexible member of the first and second pluralities of flexible members includes a distal end portion defined between the pivot axis and the second terminal end, and wherein the distal end portion is bent outwardly relative to a remainder of the flexible member and relative to the outer portion of the flexible member.
5. The clutch device of any preceding example, further comprising:
   a third plurality of flexible members;
   a fourth plurality of flexible members, wherein the first and third pluralities of flexible members define a first stack of flexible members and the second and fourth pluralities of flexible members define a second stack of flexible members, wherein the third plurality of flexible members are interleaved with the fourth plurality of flexible members;
   at least one overlapping region defined in an area in which flexible members of each of the third and fourth pluralities of flexible members overlap each other; and
   a plurality of non-overlapping regions defined in areas on opposing sides of the overlapping region, the plurality of non-overlapping regions being regions in which the flexible members of each of the third and fourth pluralities of flexible members do not overlap each other,
   wherein, in the locked arrangement of the clutch device, an outer portion of each of the plurality of non-overlapping regions extends at a third angle relative to a plane defined by a corresponding overlapping region of the at least one overlapping region such that the third and fourth pluralities of flexible members engage and interlock with each other and prevent the third and fourth pluralities of flexible members from sliding with respect to each other, and
   wherein the outer portions of each of the plurality of non-overlapping regions are configured to be moved to a fourth angle relative to the plane defined by at least one overlapping region to bend the flexible member outwardly, the fourth angle being different than the third angle, and such movement causing the clutch device to move into the unlocked arrangement in which the third and fourth pluralities of flexible members are disengaged from each other so as to allow the third and fourth pluralities of flexible members to sliding with respect to each other.
6. The clutch device of example 5, further comprising:
   at least one first cord extending through the first and third pluralities of flexible members; and
   at least one second cord extending through the second and fourth pluralities of flexible members,
   wherein the first and third pluralities of flexible members are configured to be pulled toward each other and subsequently held in tension via the at least one first cord so as to form the first stack of flexible members, and
   wherein the second and fourth pluralities of flexible members are configured to be pulled toward each other and subsequently held in tension via at least one second cord so as to form the second stack of flexible members.
7. The clutch device of example 6, wherein the at least one first and second cords extends through the first, second, third, and fourth pluralities of flexible members adjacent to the pivot axis.
8 The clutch device of any of examples 5 to 7, further comprising:
   a first clamp extending around the distal end portions of the flexible members of the first and third pluralities of flexible members; and
   a second clamp extending around the distal end portions of the flexible members of the second and fourth pluralities of flexible members,
   wherein the first and second clamps are configured to clamp the second terminal ends of the flexible members together so as to cause the respective pluralities of flexible members to rotate and move the clutch device into the unlocked arrangement.
9. The clutch device of any of examples 5 to 8, further comprising:
   a first bendable actuation member, wherein each flexible member of the first and third pluralities of flexible members is coupled to the first bendable actuation member; and
   a second bendable actuation member, wherein each flexible member of the second and fourth pluralities of flexible members is coupled to the second bendable actuation member.
10. The clutch device of example 9, wherein the first and second bendable actuation members are configured to be bent so as to move opposing outer ends of the first and second bendable actuation members in a direction away from the overlapping region to cause the respective pluralities of flexible members to rotate and move the clutch device into the unlocked arrangement.
11. A clutch device, comprising:
   a first plurality of flexible members; and
   a second plurality of flexible members interleaved with the first plurality of flexible members so as to define an overlapping region in an area in which flexible members of the first and second pluralities of flexible members overlap each other,
   wherein, in a locked arrangement of the clutch device, subject to an external load, the first and second pluralities of flexible members exert a force on the overlapping region so as to cause the first and second pluralities of flexible members to interlock with each other, and
   wherein the first and second pluralities of flexible members are configured to be moved so as to remove the force exerted on the overlapping region and move the clutch device into an unlocked arrangement in which the first and second pluralities of flexible members are disengaged from each other.
12. The clutch device of example 11,
   wherein non-overlapping regions are defined in areas of the first and second pluralities of flexible members in which the flexible members do not overlap each other, and
   wherein, in a locked arrangement of the clutch device, the first and second non-overlapping regions exert a force on the overlapping region so as to cause the first and second pluralities of flexible members to interlock with each other and prevent the first and second pluralities of flexible members from sliding with respect to each other.
13. The clutch device of example 12,
   wherein, in the locked arrangement of the clutch device, an outer portion of each non-overlapping region of each flexible member extends at a first angle relative to a plane defined by a corresponding overlapping region of the flexible member, and
   wherein the first and second non-overlapping regions of the first and second pluralities of flexible members are configured to be rotated outwardly relative to the overlapping region so as to increase the first angle, remove the force exerted on the overlapping region, and move the clutch device into the unlocked arrangement in which the first and second pluralities of flexible members are disengaged from each other to allow the first and second pluralities of flexible members to slide with respect to each other.
14. The clutch device of example 13,
   wherein each flexible member of the first and second pluralities of flexible members includes a first terminal end that overlaps an opposing flexible member of the other of the first and second pluralities of flexible members,
   wherein each flexible member of the first and second pluralities of flexible members further includes a second terminal end opposite the first terminal end, and
   wherein a pivot axis is defined on each flexible member of the first and second pluralities of flexible members about which the respective plurality of flexible members is configured to be rotated.
15. The clutch device of example 14,
   wherein each flexible member of the first and second pluralities of flexible members includes a distal end portion defined between the pivot axis and the second terminal end, and
   wherein the distal end portion is bent outwardly relative to a remainder of the flexible member and relative the outer portion of the flexible member.
16. The clutch device of example 14 or 15, further comprising:
   at least one first cord coupled to the first plurality of flexible members closer to the second terminal end than the first terminal end of each flexible member; and
   at least one second cord coupled to the second plurality of flexible members closer to the second terminal end than the first terminal end of each flexible member,
   wherein the at least one first and second cords are interwoven with each flexible member of the respective first and second pluralities of flexible members and are configured to be tensioned so as to compress the first and second pluralities of flexible members together.
17. The clutch device of any of examples 11 to 16, further comprising:
   a first bendable actuation member, wherein the first plurality of flexible members is coupled to the first bendable actuation member; and
   a second bendable actuation member, wherein the second plurality of flexible members is coupled to the second bendable actuation member,
   wherein the first and second bendable actuation members are configured to be bent so as to move opposing outer ends of the first and second bendable actuation members in a direction away from the overlapping region to cause the first and second pluralities of flexible members to rotate and move the clutch device into the unlocked arrangement.
18. The clutch device of example 17,
   wherein a number of flexible members of the first and second pluralities of flexible members that are disengaged when the first bendable actuation member is bent such that the opposing outer ends move in a direction away from the overlapping region depends on an extent of the bending of the first bendable actuation member, and
   wherein the extent of the bending of the first bendable actuation member is selectable such that the number of flexible members that are disengaged when the first bendable actuation member is bent is selectable such that a number of flexible members that remain engaged after bending of the first bendable actuation member is also selectable.
19. The clutch device of any of examples 11 to 18, further comprising:
   a first resetting cord member having a first end coupled to the first plurality of flexible members and a second end opposite the first end coupled to the second plurality of flexible members and configured to prevent the first and second pluralities of flexible members from sliding away from each other beyond a predetermined stack separation distance.
20. The clutch device of example 19, wherein the first resetting cord member is resilient such that the first resetting cord member moves the first and second pluralities of flexible members toward each other after the first and second pluralities of flexible members have been slid away from each other.
21. A method of selectively locking and unlocking a clutch device, comprising:
   applying a force to at least one of an outer portion of a first plurality of flexible members of a clutch device or an outer portion of a second plurality of flexible members of the clutch device, the first and second pluralities of flexible members being interleaved with respect to each other to cause the interleaved flexible members of the first and second pluralities of flexible members to move from a locked arrangement, in which the interleaved flexible members are substantially locked with respect to each other, to an unlocked arrangement, in which the interleaved flexible members of the first and second pluralities of flexible members are disengaged from each other.
22. The method of example 21, further comprising:
   interleaving the second plurality of flexible members with the first plurality of flexible members so as to define at least one overlapping region in an area in which flexible members of the first and second pluralities of flexible members overlap each other,
   wherein, in a locked arrangement of the clutch device, the first and second pluralities of flexible members exert a force on the at least one overlapping region to cause the first and second pluralities of flexible members to interlock with each other.
23. The method of example 22,
   wherein a plurality of non-overlapping regions of the first and second pluralities of flexible members are defined in areas on opposing sides of the overlapping region, and
   wherein the applying of the force to cause the interleaved flexible members of the first and second pluralities of flexible members to move to the unlocked arrangement includes moving outer portions of each of the plurality of non-overlapping regions to a second angle relative to a plane defined by the at least one overlapping region to bend the flexible member outwardly, the second angle being different than the first angle, and such movement causing the clutch device to move into the unlocked arrangement.
24. The method of example 23, wherein moving the outer portions of each of the plurality of non-overlapping regions includes rotating the outer portions.
25. The clutch device of example 24,
   wherein each flexible member of the first and second pluralities of flexible members includes a distal end portion defined between a pivot axis and a terminal end of the flexible member,
   wherein the distal end portion is bent outwardly relative to a remainder of the flexible member and relative to the outer portion of the flexible member, and
   wherein the rotating of the outer portions is effected by movement of the distal end portions which causes rotation of the first and second pluralities of flexible members about the respective pivot axis.
26. The method of any of examples 22 to 25, wherein the interleaving of the second plurality of flexible members with the first plurality of flexible members includes sequentially and alternatingly stacking respective flexible members of the first and second pluralities of flexible members so as to form an interleaved stack of first and second flexible members.
27. The method of any of examples 21 to 25, further comprising:
   forming the first and second pluralities of flexible members via at least one of additive manufacturing or 3D printing.

It should be understood that the subject matter defined in the appended claims is not necessarily limited to the specific implementations described above. The specific implementations described above are disclosed as examples only. One skilled in the art will appreciate further features and advantages of the disclosure based on the above-described embodiments. Accordingly, the disclosure is not to be limited by what has been particularly shown and described, except as indicated by the appended claims. For example, while the present embodiments often include a single feature, it is possible that multiple of the same features can be incorporated into the design of a clutch device similar to those described above without departing from the spirit of the present disclosure. For example, although not explicitly described herein, the features regarding the bendable actuation members could be utilized with the half-stack arrangement. Any and all combinations of features envisioned by a person skilled in the art based on the present disclosure and knowledge in the art are contemplated by the description herein.

Some non-limiting claims that are supported by the contents of the present disclosure are provided below.

What is claimed is:

1. A clutch device, comprising:
a first plurality of flexible members;
a second plurality of flexible members interleaved with the first plurality of flexible members;
at least one overlapping region defined in an area in which flexible members of each of the first and second pluralities of flexible members overlap each other; and
a plurality of non-overlapping regions defined in areas on opposing sides of the overlapping region, the plurality of non-overlapping regions being regions in which the flexible members of each of the first and second pluralities of flexible members do not overlap each other,
wherein, in a locked arrangement of the clutch device, an outer portion of each of the plurality of non-overlapping regions extends at a first angle relative to a plane defined by a corresponding overlapping region of the at least one overlapping region such that the first and second pluralities of flexible members engage and interlock with each other and prevent the first and second pluralities of flexible members from sliding with respect to each other, and
wherein the outer portions of each of the plurality of non-overlapping regions are configured to be moved to a second angle relative to the plane defined by the at least one overlapping region to bend the flexible member outwardly, the second angle being different than the first angle, and such movement causing the clutch device to move into an unlocked arrangement in which the first and second pluralities of flexible members are disengaged from each other so as to allow the first and second pluralities of flexible members to slide with respect to each other.

2. The clutch device of claim 1,
wherein each flexible member of the first and second pluralities of flexible members includes a first terminal end that overlaps an opposing flexible member of the other of the first and second pluralities of flexible members,
wherein each flexible member of the first and second pluralities of flexible members further includes a second terminal end opposite the first terminal end, and
wherein a pivot axis is defined on each flexible member of the first and second pluralities of flexible members about which the respective plurality of flexible members is configured to be rotated.

3. The clutch device of claim 2, wherein each flexible member of the first and second pluralities of flexible members includes a distal end portion defined between the pivot axis and the second terminal end, and wherein the distal end portion is bent outwardly relative to a remainder of the flexible member and relative to the outer portion of the flexible member.

4. The clutch device of claim 3, further comprising:
a third plurality of flexible members;
a fourth plurality of flexible members, wherein the first and third pluralities of flexible members define a first stack of flexible members and the second and fourth pluralities of flexible members define a second stack of flexible members, wherein the third plurality of flexible members are interleaved with the fourth plurality of flexible members;
at least one overlapping region defined in an area in which flexible members of each of the third and fourth pluralities of flexible members overlap each other; and
a plurality of non-overlapping regions defined in areas on opposing sides of the overlapping region, the plurality of non-overlapping regions being regions in which the flexible members of each of the third and fourth pluralities of flexible members do not overlap each other,
wherein, in the locked arrangement of the clutch device, an outer portion of each of the plurality of non-overlapping regions extends at a third angle relative to a plane defined by a corresponding overlapping region of the at least one overlapping region such that the third and fourth pluralities of flexible members engage and interlock with each other and prevent the third and fourth pluralities of flexible members from sliding with respect to each other, and
wherein the outer portions of each of the plurality of non-overlapping regions are configured to be moved to a fourth angle relative to the plane defined by at least one overlapping region to bend the flexible member outwardly, the fourth angle being different than the third angle, and such movement causing the clutch device to move into the unlocked arrangement in which the third and fourth pluralities of flexible members are disengaged from each other so as to allow the third and fourth pluralities of flexible members to sliding with respect to each other.

5. The clutch device of claim 4, further comprising:
at least one first cord extending through the first and third pluralities of flexible members; and
at least one second cord extending through the second and fourth pluralities of flexible members,
wherein the first and third pluralities of flexible members are configured to be pulled toward each other and subsequently held in tension via the at least one first cord so as to form the first stack of flexible members, and
wherein the second and fourth pluralities of flexible members are configured to be pulled toward each other and subsequently held in tension via at least one second cord so as to form the second stack of flexible members.

6. The clutch device of claim 5, further comprising:
a first clamp extending around the distal end portions of the flexible members of the first and third pluralities of flexible members; and
a second clamp extending around the distal end portions of the flexible members of the second and fourth pluralities of flexible members,
wherein the first and second clamps are configured to clamp the second terminal ends of the flexible members together so as to cause the respective pluralities of flexible members to rotate and move the clutch device into the unlocked arrangement.

7. The clutch device of claim 5, further comprising:
a first bendable actuation member, wherein each flexible member of the first and third pluralities of flexible members is coupled to the first bendable actuation member; and
a second bendable actuation member, wherein each flexible member of the second and fourth pluralities of flexible members is coupled to the second bendable actuation member.

8. The clutch device of claim 7, wherein the first and second bendable actuation members are configured to be bent so as to move opposing outer ends of the first and second bendable actuation members in a direction away from the overlapping region to cause the respective pluralities of flexible members to rotate and move the clutch device into the unlocked arrangement.

9. A clutch device, comprising:
a first plurality of flexible members; and
a second plurality of flexible members interleaved with the first plurality of flexible members so as to define an overlapping region in an area in which flexible members of the first and second pluralities of flexible members overlap each other,
wherein, in a locked arrangement of the clutch device, subject to an external load, the first and second pluralities of flexible members exert a force on the overlapping region so as to cause the first and second pluralities of flexible members to interlock with each other, and
wherein the first and second pluralities of flexible members are configured to be moved so as to remove the force exerted on the overlapping region and move the clutch device into an unlocked arrangement in which the first and second pluralities of flexible members are disengaged from each other.

10. The clutch device of claim 9,
wherein non-overlapping regions are defined in areas of the first and second pluralities of flexible members in which the flexible members do not overlap each other, and
wherein, in a locked arrangement of the clutch device, the first and second non-overlapping regions exert a force on the overlapping region so as to cause the first and second pluralities of flexible members to interlock with each other and prevent the first and second pluralities of flexible members from sliding with respect to each other.

11. The clutch device of claim 10,
wherein, in the locked arrangement of the clutch device, an outer portion of each non-overlapping region of each flexible member extends at a first angle relative to a plane defined by a corresponding overlapping region of the flexible member, and
wherein the first and second non-overlapping regions of the first and second pluralities of flexible members are configured to be rotated outwardly relative to the overlapping region so as to increase the first angle, remove the force exerted on the overlapping region, and move the clutch device into the unlocked arrangement in which the first and second pluralities of flexible members are disengaged from each other to allow the first and second pluralities of flexible members to slide with respect to each other.

12. The clutch device of claim 11,
wherein each flexible member of the first and second pluralities of flexible members includes a first terminal end that overlaps an opposing flexible member of the other of the first and second pluralities of flexible members,
wherein each flexible member of the first and second pluralities of flexible members further includes a second terminal end opposite the first terminal end, and
wherein a pivot axis is defined on each flexible member of the first and second pluralities of flexible members about which the respective plurality of flexible members is configured to be rotated.

13. The clutch device of claim 12,
wherein each flexible member of the first and second pluralities of flexible members includes a distal end portion defined between the pivot axis and the second terminal end, and
wherein the distal end portion is bent outwardly relative to a remainder of the flexible member and relative the outer portion of the flexible member.

14. The clutch device of claim 12, further comprising:
a first bendable actuation member, wherein the first plurality of flexible members is coupled to the first bendable actuation member; and
a second bendable actuation member, wherein the second plurality of flexible members is coupled to the second bendable actuation member,
wherein the first and second bendable actuation members are configured to be bent so as to move opposing outer ends of the first and second bendable actuation members in a direction away from the overlapping region to cause the first and second pluralities of flexible members to rotate and move the clutch device into the unlocked arrangement.

15. The clutch device of claim 12, further comprising:
a first resetting cord member having a first end coupled to the first plurality of flexible members and a second end opposite the first end coupled to the second plurality of flexible members and configured to prevent the first and second pluralities of flexible members from sliding away from each other beyond a predetermined stack separation distance.

16. A method of selectively locking and unlocking a clutch device, comprising:
applying a force to at least one of an outer portion of a first plurality of flexible members of a clutch device or an outer portion of a second plurality of flexible members of the clutch device, the first and second pluralities of flexible members being interleaved with respect to each other to cause the interleaved flexible members of the first and second pluralities of flexible members to move from a locked arrangement, in which the interleaved flexible members are substantially locked with respect to each other, to an unlocked arrangement, in which the interleaved flexible members of the first and second pluralities of flexible members are disengaged from each other.

17. The method of claim 16, further comprising:
interleaving the second plurality of flexible members with the first plurality of flexible members so as to define at least one overlapping region in an area in which flexible members of the first and second pluralities of flexible members overlap each other,
wherein, in a locked arrangement of the clutch device, the first and second pluralities of flexible members exert a force on the at least one overlapping region to cause the first and second pluralities of flexible members to interlock with each other.

18. The method of claim 17,
wherein a plurality of non-overlapping regions of the first and second pluralities of flexible members are defined in areas on opposing sides of the overlapping region, and
wherein the applying of the force to cause the interleaved flexible members of the first and second pluralities of flexible members to move to the unlocked arrangement includes moving outer portions of each of the plurality of non-overlapping regions to a second angle relative to a plane defined by the at least one overlapping region to bend the flexible member outwardly, the second angle being different than the first angle, and such movement causing the clutch device to move into the unlocked arrangement.

19. The method of claim 18, wherein moving the outer portions of each of the plurality of non-overlapping regions includes rotating the outer portions.

20. The method of claim 19, further comprising:
forming the first and second pluralities of flexible members via at least one of additive manufacturing or 3D printing.
